(19) United States
(12) Patent Application Publication
(10) Pub. No.: US 2023/0404957 A1
(43) Pub. Date: Dec. 21, 2023

(54) Pet Food Compositions

(71) Applicant: Hill's Pet Nutrition, Inc., Topeka, KS (US)

(72) Inventors: Matthew JACKSON, Topeka, KS (US); Laura Morgan, Saint George, KS (US); Dennis Jewell, Lawrence, KS (US)

(73) Assignee: Hill's Pet Nutrition, Inc., Topeka, KS (US)

(21) Appl. No.: 18/335,194

(22) Filed: Jun. 15, 2023

Related U.S. Application Data
(60) Provisional application No. 63/352,380, filed on Jun. 15, 2022.

(51) Int. Cl. A61K 31/20; A23K 50/40; A23K 20/158; A23K 20/147; A23K 20/163; A61K 9/00; A61P 13/12

(52) U.S. Cl. CPC A61K 31/20; A23K 50/40; A23K 20/158; A23K 20/147; A23K 20/163; A61K 9/0056; A61P 13/12

(57) ABSTRACT

Pet food compositions and methods are provided herein. The pet food composition comprising a from about 5 g/1000 kcal to about 80 g/1000 kcal of a fat including one or more medium chain triglyceride, wherein at least one medium chain triglyceride comprises an octanoate component, the at least one medium chain triglyceride comprising the octanoate component comprises about 7 wt. % or more of the total weight of fat in the pet food composition; from about 40 g/1000 kcal to about 150 g/1000 kcal of a protein; optionally, from about 5 g/1000 kcal to about 55 g/1000 kcal of a fiber; optionally, from about 3 g/1000 kcal to about 130 g/1000 kcal of a carbohydrate, where all metabolizable energy amounts are based on the total metabolizable energy of the pet food composition on a dry matter basis.

### CROSS-REFERENCE TO RELATED PATENT APPLICATIONS

This application claims priority to U.S. Appl. No. 63/352,380, titled PET FOOD COMPOSITIONS and filed Jun. 15, 2022, the content of which is incorporated herein by reference in its entirety for all purposes.

### BACKGROUND

Domestic felines are generally predisposed to renal disease, with nearly 33% of felines becoming renal impaired by the age of 10 years old. Renal disease incidence and progression is marked by distinct changes in lipid metabolism. Deteriorating capacity for lipid metabolism is evident in cats, where lipidosis occurs in kidney tissue. It is known that spillover of kidney lipid metabolism leaves signatures of disease risk in the urine and circulation.

Chronic kidney disease (CKD), also known as chronic renal disease or chronic renal failure, is a progressive loss in renal function over a period of months or years. CKD can be caused by a variety of conditions and mechanisms, and affects both humans and other mammals, in particular cats. Renal function in geriatric cats progressively declines over time, leading to end-stage disease.

Hyperphosphatemia is a well-documented sequela of CKD in cats, and has been associated with increased risks for death and progression of azotemia. Phosphate retention results from inability of the kidney to excrete sufficient phosphate to balance dietary intake. Actions of the body to maintain normophosphatemia result in secondary renal hyperparathyroidism and FGF-23 excess. These hormonal derangements prevent overt hyperphosphatemia in the early stages of CKD, but are thought to contribute to bone pathology and soft tissue calcification which together with disturbed calcium-phosphate homeostasis comprise CKD-mineral and bone disorder (CKD-MBD). FGF-23 itself is a strong predictor of survival and progression in cats with CKD. Dietary phosphate restriction is the mainstay of management of CKD in cats, and has been shown to reduce plasma phosphate, FGF-23 and PTH concentrations and improve survival.

Recently, there has been increased interest in the role of magnesium in human CKD-MBD. In addition to being an essential mineral for numerous intracellular processes, magnesium is thought to be an inhibitor of vascular calcification and release of profibrotic cytokines. Interestingly, magnesium may be involved in FGF-23 regulation, because an inverse association between these two variables was observed in human CKD patients on hemodialysis, and FGF-23 concentrations were increased in rodents fed a magnesium deficient diet. While there has been recent interest, little is known about the role of magnesium in feline CKD-MBD.

Accordingly, there is an ongoing need for pet food compositions and methods for alleviating, mitigating, or reducing symptoms of a pet suffering from a renal disease.

### BRIEF SUMMARY

This summary is intended merely to introduce a simplified summary of some aspects of one or more implementations of the present disclosure. Further areas of applicability of the present disclosure will become apparent from the detailed description provided hereinafter. This summary is not an extensive overview, nor is it intended to identify key or critical elements of the present teachings, nor to delineate the scope of the disclosure. Rather, its purpose is merely to present one or more concepts in simplified form as a prelude to the detailed description below.

In accordance with an aspect of the invention, provided is a pet food composition comprising: from about 5 g/1000 kcal to about 80 g/1000 kcal of a fat comprising one or more medium chain triglyceride, wherein at least one medium chain triglyceride comprises an octanoate component, the at least one medium chain triglyceride comprising the octanoate component comprises about 7% or more of the total weight of fat in the pet food composition; from about 40 g/1000 kcal to about 150 g/1000 kcal of a protein; optionally, from about 5 g/1000 kcal to about 55 g/1000 kcal of a fiber; and optionally, from about 3 g/1000 kcal to about 130 g/1000 kcal of a carbohydrate, wherein all metabolizable energy amounts are based on the total metabolizable energy of the pet food composition on a dry matter basis.

According to another aspect, provided is a method for alleviating, mitigating, or reducing symptoms of a pet suffering from a renal disease. The method may comprise increasing the ratio of circulating albumin to globulin in the pet by administering a pet food composition disclosed herein to the pet. In some instances, the method may include providing and/or feeding the canine the pet food compositions for 1 or more days, preferably 5 or more days, preferably 7 or more days, preferably 10 or more days, preferably 14 or more days, preferably 30 or more days, or preferably 42 or more days. The method may include feeding the pet one time a day, two times a day, three times a day, or in some embodiments four or more times a day.

In accordance with a further aspect, provided is a method for alleviating, mitigating, or reducing symptoms of a pet suffering from a renal disease comprising: diagnosing a pet with renal disease by measuring serum albumin in the pet; and increasing the ratio of circulating albumin to globulin in the pet by administering an amount of a pet food composition disclosed herein to the pet. In some embodiments, the method further comprises measuring serum albumin in the pet after one or more weeks of the pet being fed the amount of pet food composition, and instructing an increase or decrease in the amount of pet food composition consumed by the pet.

Preferably, the pet food compositions and methods disclosed herein produce an increase of about 5% or more, about 10% or more, about 15% or more, about 20% or more, or about 25% or more, of at least one long-chain acylcarnitine, osmotic regulator, phospholipid, sphingolipid, endocannabinoid, or a combination of two or more thereof. The increase in the one or more long-chain acylcarnitine, osmotic regulator, phospholipid, sphingolipid, and/or endocannabinoid may be achieved after about 3 days post-ingestion with the pet consuming such pet food composition at least once a day (e.g., twice or three times a day) in an amount based on the desired and/or recommended daily nutritional intake for the pet for 14 days.

The pet food compositions and methods may also increase the molar ratio of circulating albumin to globulin in a pet consuming the pet food composition. In some embodiments, the pet food compositions and/or methods disclosed herein may increase the molar ratio of circulating albumin to globulin by about 3% or more, about 6% or more, about 9% or more, about 14% or more, about 17% or more, about 22% or more, about 27% or more. The increase in the molar ratio of circulating albumin to globulin may be achieved after about 3 days post-ingestion by a pet consuming such pet food composition at least once a day (e.g., twice or three times a day) in an amount based on the desired and/or recommended daily nutritional intake for the pet for 14 days.

Preferably, the pet food compositions and method disclosed herein maintain both a blood urea concentration and/or a creatinine concentration within a 10±% range (e.g., within a 7±% range or 5±% range). The maintenance of the urea concentration and/or creatinine concentration may be obtained up to and/or after about 3 days post-ingestion by a pet, with such the pet being fed the pet food composition at least once a day (e.g., twice or three times a day) an amount based on the desired and/or recommended daily nutritional intake for the pet for 14 days.

### DETAILED DESCRIPTION

For illustrative purposes, the principles of the present invention are described by referencing various exemplary embodiments thereof. Although certain embodiments of the invention are specifically described herein, one of ordinary skill in the art will readily recognize that the same principles are equally applicable to, and can be employed in other compositions and methods. Before explaining the disclosed embodiments of the present invention in detail, it is to be understood that the invention is not necessarily limited in its application to the details of any particular embodiment disclosed. The terminology used herein is for the purpose of description and not of limitation.

As used herein and in the appended claims, the singular forms “a”, “an”, and “the” include plural references unless the context dictates otherwise. The singular form of any class of the ingredients refers not only to one ingredient within that class, but also to a mixture of those ingredients. The terms “a” (or “an”), “one or more” and “at least one” may be used interchangeably herein. The terms “comprising”, “including”, and “having” may be used interchangeably. The term “include” should be interpreted as “include, but are not limited to”. The term “including” should be interpreted as “including, but are not limited to”.

As used throughout, ranges are used as shorthand for describing each and every value that is within the range. Any value within the range can be selected as the terminus of the range. Thus, a range from 1-5, includes specifically 1, 2, 3, 4 and 5, as well as subranges such as 2-5, 3-5, 2-3, 2-4, 1-4, etc. The term “about” when referring to a number means any number within a range of 10% of the number. For example, the phrase “about 2.0 wt. %” refers to a number between and including 1.8 wt. % and 2.2 wt. %.

All references cited herein are hereby incorporated by reference in their entireties. In the event of a conflict in a definition in the present disclosure and that of a cited reference, the present disclosure controls.

The abbreviations and symbols as used herein, unless indicated otherwise, take their ordinary meaning. The abbreviation “wt. %” means percent by weight with respect to the pet food composition. The symbol “°” refers to a degree, such as a temperature degree or a degree of an angle. The symbols “h”, “min”, “mL”,” nm”, “μm” means hour, minute, milliliter, nanometer, and micrometer, respectively. The abbreviation “UV-VIS” referring to a spectrometer or spectroscopy, means Ultraviolet-Visible. The abbreviation “rpm” means revolutions per minute.

Any member in a list of species that are used to exemplify or define a genus, may be mutually different from, or overlapping with, or a subset of, or equivalent to, or nearly the same as, or identical to, any other member of the list of species. Further, unless explicitly stated, such as when reciting a Markush group, the list of species that define or exemplify the genus is open, and it is given that other species may exist that define or exemplify the genus just as well as, or better than, any other species listed.

All components and elements positively set forth in this disclosure can be negatively excluded from the claims. In other words, the pet food compositions of the instant disclosure can be free or essentially free of all components and elements positively recited throughout the instant disclosure. In some instances, the pet food compositions of the present disclosure may be substantially free of non-incidental amounts of the ingredient(s) or compound(s) described herein. A non-incidental amount of an ingredient or compound is the amount of that ingredient or compound that is added into the pet food composition by itself. For example, a pet food composition may be substantially free of a non-incidental amount of an ingredient or compound, although such ingredient(s) or compound(s) may be present as part of a raw material that is included as a blend of two or more compounds. Substantially free, unless other defined or described herein, typically refers to an ingredient or compound in an amount of about 2 wt. % or less, about 1.5 wt. % or less, about 1 wt. % or less, about 0.5 wt. % or less, about 0.1 wt. % or less, or about 0.05 wt. % or less, or about 0.01 wt. % or less, based on the total weight of the pet food composition on a dry matter basis.

Some of the various categories of components identified may overlap. In such cases where overlap may exist and the pet food composition includes both components (or the composition includes more than two components that overlap), an overlapping compound does not represent more than one component. For example, certain components or ingredients may be characterized as both an ancient grain and an amaranth. If a particular pet food care composition recites both an ancient grain and an amaranth, a compound that may be characterized as both an ancient grain and an amaranth will serve only as either an ancient grain or an amaranth—not both.

As used herein, the term “pet” could be used interchangeably with “companion animal” and refers to an animal of any species kept by a caregiver as a pet or any animal of a variety of species that have been widely domesticated as pets, including canines (Canis familiaris) and felines (Felis domesticus). Thus, a pet may include but is not limited to, working dogs, pet dogs, cats kept for rodent control (i.e. farm cats), pet cats, ferrets, birds, reptiles, rabbits, and fish.

To the extent that food and food ingredient contain water/moisture, the dry matter represents everything in the sample other than water including, for example, protein, fiber, fat, carbohydrates, minerals, etc. Dry matter weight is the total weight minus the weight of any water. The skilled artisan would readily recognize and understand nutritional amounts and percentages expressed as dry matter amounts, dry matter weights and dry matter percentages.

Dry matter intake per day is calculated as the total nutritional intake per day excluding all water. For example, an amount of an ingredient equal to a specific percent of daily nutritional intake refers to the amount of that ingredient in dry matter form (i.e., excluding all water) relative to the total amount of dry matter consumed (also excluding all water) in a day.

“Daily nutritional intake” and “total nutritional intake per day” refer to dry matter intake per day. That is, water weight is not included in calculating the amount of nutrition consumed per day. To calculate percent of an ingredient of total daily intake on a dry matter basis, water is removed from the total intake to give total daily dry matter intake and the percentage of the ingredient is based on amount of ingredient present as dry matter.

“Metabolizable energy” as used herein refers to apparent metabolizable energy and means the gross energy of the feed consumed minus the gross energy contained in the faeces, urine, and gaseous products of digestion. Energy digestibility and metabolizable energy may be measured as the difference between the intake of gross energy and the gross energy excreted in the faeces or the digesta present in specified segment of the gastro-intestinal tract using the same methods to measure the digestibility of nutrients, with appropriate corrections for nitrogen excretion to calculate metabolizable energy of feed. Metabolizable energy values may be determined by methods known by those skilled in the art, such as detailed in Association of American Feed Control Officials: Official Publication, Atlanta, Ga., pages 160-165 (2006), which is incorporated herein by reference in its entirety for all purposes.

As used herein, an “ingredient” refers to any component of a pet food composition. The term “nutrient” refers to a substance that provides nourishment and thus has a nutrient value. In some cases, an ingredient may comprise more than one “nutrient,” for example, a composition may comprise corn comprising important nutrients including both protein and carbohydrate.

Aspects of the invention are directed to pet food compositions and methods of use thereof. The pet food compositions disclosed herein preferably provide alleviation, mitigation, or a reduction of symptoms for a pet suffering from a renal disease. The inventors discovered certain embodiments of invention can improve lipid markers of renal health and disease risk, including long-chain acylcarnitines, osmotic regulators, phospholipids, sphingolipids and endocannabinoids, and the incorporation of the anti-inflammatory long chain polyunsaturated docosahexaenoate (22:6n3) into phospholipids. The inventors also discovered that certain pet food compositions may desirably be formulated to provide an increase in the ratio of circulating albumin to globulin while maintaining blood urea and creatinine. In some preferred embodiments, the increase in the ratio of circulating albumin to globulin is achieved by both increasing the amount of albumin and decreasing the amount of globulin.

In accordance with an aspect of the invention, provided is a pet food composition comprising: a fat comprising one or more medium chain triglyceride, wherein at least one medium chain triglyceride comprises an octanoate component, the at least one medium chain triglyceride comprising the octanoate component comprises about 7 wt. % or more of the fat, based on the total weight of the pet food composition; from about 40 g/1000 kcal to about 150 g/1000 kcal of a protein; optionally, from about 5 g/1000 kcal to about 55 g/1000 kcal of a fiber; and optionally, from about 3 g/1000 kcal to about 130 g/1000 kcal of a carbohydrate, wherein all metabolizable energy amounts are based on the total metabolizable energy of the pet food composition on a dry matter basis.

Preferably, the pet food composition produces an increase of about 5% or more, about 10% or more, about 15% or more, about 20% or more, or about 25% or more, of at least one long-chain acylcarnitine, osmotic regulator, phospholipid, sphingolipid, endocannabinoid, or a combination of two or more thereof. The pet food compositions may also increase the incorporation of anti-inflammatory long chain polyunsaturated docosahexaenoate (22:6n3) into phospholipids by about 5% or more, about 10% or more, about 15% or more, about 20% or more, or about 25% or more. The increase in the one or more long-chain acylcarnitines, osmotic regulators, phospholipids, sphingolipids, endocannabinoids, and/or increase in the incorporation of anti-inflammatory long chain polyunsaturated docosahexaenoate (22:6n3) into phospholipids may be achieved after about 3 days post-ingestion by a pet consuming the pet food composition in an amount based on the desired and/or recommended daily nutritional intake for the pet for 14 days. The recommended daily nutritional intake may be based on the Association of American Feed Control Officials (AAFCO) recommendations for the pet. The desired and/or recommended amount of pet food compositions consumed by the pet may be provided as one, two, three, or more servings throughout the day.

Additionally or alternatively, the pet food compositions may provide an increase in the ratio of circulating albumin to globulin by both increasing the amount of albumin and/or decreasing the amount of globulin. In some embodiments, the pet food compositions may increase the molar ratio of circulating albumin to globulin by about 3% or more, about 6% or more, about 9% or more, about 14% or more, about 17% or more, about 22% or more, about 27% or more. The increase in the molar ratio of circulating albumin to globulin may be achieved after about 3 days post-ingestion by a pet consuming the pet food composition in an amount based on the desired and/or recommended daily nutritional intake for the pet for 14 days. The recommended daily nutritional intake may be based on the Association of American Feed Control Officials (AAFCO) recommendations for the pet. The desired and/or recommended amount of pet food compositions consumed by the pet may be provided as one, two, three, or more servings throughout the day.

Preferably, the pet food compositions maintains both a blood urea concentration and a creatinine concentration within a 10±% range (e.g., within a 7±% range or 5±% range) after about 3 days post-ingestion by a pet consuming the pet food composition in an amount based on the desired and/or recommended daily nutritional intake for the pet for 14 days. As noted above, the recommended daily nutritional intake may be based on the Association of American Feed Control Officials (AAFCO) recommendations for the pet. The desired and/or recommended amount of pet food compositions consumed by the pet may be provided as one, two, three, or more servings throughout the day.

Suitable components, such as those listed below, may be included or excluded from the formulations for the pet food compositions depending on the specific combination of other ingredients and the form of the pet food compositions. In some embodiments, the pet food compositions disclosed herein may be in the form of a stand-alone pet food, as a supplement to pet food, as a pet food treat, or the like.

The term “fat” generally refers to a lipid or mixture of lipids that may generally be a solid or a liquid at ordinary room temperatures (e.g., 25° C.) and pressures (e.g., 1 atm). In some instances, the fat may be a viscous liquid or an amorphous solid at standard room temperature and pressure.

The pet food composition may be formulated to include about 5 g/1000 kcal to about 80 g/1000 kcal or about 10 g/1000 kcal to about 80 g/1000 kcal of fat, based on the total metabolizable energy of the pet food composition on a dry matter basis. For instance, the fat may be present in the pet food composition in an amount from about 5 g/1000 kcal to about 75 g/1000 kcal, about 5 g/1000 kcal to about 60 g/1000 kcal, about 5 g/1000 kcal to about 50 g/1000 kcal, about 5 g/1000 kcal to about 40 g/1000 kcal, about 5 g/1000 kcal to about 30 g/1000 kcal, about 5 g/1000 kcal to about 25 g/1000 kcal, about 5 g/1000 kcal to about 20 g/1000 kcal; from about 10 g/1000 kcal to about 75 g/1000 kcal, about 10 g/1000 kcal to about 60 g/1000 kcal, about 10 g/1000 kcal to about 50 g/1000 kcal, about 10 g/1000 kcal to about 40 g/1000 kcal, about 10 g/1000 kcal to about 30 g/1000 kcal, about 10 g/1000 kcal to about 25 g/1000 kcal, about 10 g/1000 kcal to about 20 g/1000 kcal; from about 16 g/1000 kcal to about 80 g/1000 kcal, about 16 g/1000 kcal to about 75 g/1000 kcal, about 16 g/1000 kcal to about 60 g/1000 kcal, about 16 g/1000 kcal to about 50 g/1000 kcal, about 16 g/1000 kcal to about 40 g/1000 kcal, about 16 g/1000 kcal to about 30 g/1000 kcal, about 16 g/1000 kcal to about 25 g/1000 kcal; from about 22.5 g/1000 kcal to about 80 g/1000 kcal, about 22.5 g/1000 kcal to about 75 g/1000 kcal, about 22.5 g/1000 kcal to about 60 g/1000 kcal, about 22.5 g/1000 kcal to about 50 g/1000 kcal, about 22.5 g/1000 kcal to about 40 g/1000 kcal, about 22.5 g/1000 kcal to about 30 g/1000 kcal; from about 30 g/1000 kcal to about 80 g/1000 kcal, about 30 g/1000 kcal to about 75 g/1000 kcal, about 30 g/1000 kcal to about 60 g/1000 kcal, about 30 g/1000 kcal to about 50 g/1000 kcal, about 30 g/1000 kcal to about 40 g/1000 kcal; from about 40 g/1000 kcal to about 80 g/1000 kcal, about 40 g/1000 kcal to about 75 g/1000 kcal, about 40 g/1000 kcal to about 60 g/1000 kcal, about 40 g/1000 kcal to about 50 g/1000 kcal; from about 50 g/1000 kcal to about 80 g/1000 kcal, about 50 g/1000 kcal to about 75 g/1000 kcal, about 50 g/1000 kcal to about 60 g/1000 kcal; from about 60 g/1000 kcal to about 80 g/1000 kcal, about 60 g/1000 kcal to about 75 g/1000 kcal; from about 65 g/1000 kcal to about 80 g/1000 kcal, about 65 g/1000 kcal to about 75 g/1000 kcal; about 70 g/1000 kcal to about 80 g/1000 kcal, or 75 g/1000 kcal to about 80 g/1000 kcal, including ranges and subranges thereof, based on the total metabolizable energy of the pet food composition on a dry matter basis.

The fat in the pet food composition typically comprises one or more medium chain triglyceride(s), with at least one medium chain triglyceride having an octanoate component. The pet food compositions are generally formulated such that the medium chain triglyceride(s) having an octanoate component comprise about 7% or more of the total weight of the fat in the pet food composition. For example, the total amount of fat in the pet food composition may be comprised of medium chain triglyceride(s) in an amount of about 7 to about 60 wt. %, about 7 to about 50 wt. %, about 7 to about 45 wt. %, about 7 to about 40 wt. %, about 7 to about 37 wt. %, about 7 to about 34 wt. %, about 7 to about 31 wt. %, about 7 to about 28 wt. %, about 7 to about 25 wt. %, about 7 to about 22 wt. %, about 7 to about 17 wt. %, about 7 to about 12 wt. %; from about 12 to about 60 wt. %, about 12 to about 50 wt. %, about 12 to about 45 wt. %, about 12 to about 40 wt. %, about 12 to about 37 wt. %, about 12 to about 34 wt. %, about 12 to about 31 wt. %, about 12 to about 28 wt. %, about 12 to about 25 wt. %, about 12 to about 22 wt. %, about 12 to about 17 wt. %; from about 17 to about 60 wt. %, about 17 to about 50 wt. %, about 17 to about 45 wt. %, about 17 to about 40 wt. %, about 17 to about 37 wt. %, about 17 to about 34 wt. %, about 17 to about 31 wt. %, about 17 to about 28 wt. %, about 17 to about 25 wt. %; from about 23 to about 60 wt. %, about 23 to about 50 wt. %, about 23 to about 45 wt. %, about 23 to about 40 wt. %, about 23 to about 37 wt. %, about 23 to about 34 wt. %, about 23 to about 31 wt. %; from about 27 to about 60 wt. %, about 27 to about 50 wt. %, about 27 to about 45 wt. %, about 27 to about 40 wt. %, about 27 to about 37 wt. %, about 27 to about 34 wt. %, about 27 to about 31 wt. %; from about 31 to about 60 wt. %, about 31 to about 50 wt. %, about 31 to about 45 wt. %, about 31 to about 40 wt. %, about 31 to about 37 wt. %; from about 35 to about 60 wt. %, about 35 to about 50 wt. %, about 35 to about 45 wt. %, about 35 to about 40 wt. %; from about 39 to about 60 wt. %, about 39 to about 50 wt. %, about 39 to about 45 wt. %; from about 43 to about 60 wt. %, about 43 to about 50 wt. %, about 47 to about 60 wt. %, or any range or subrange thereof, based on the total weight of the fat in the pet food composition on a dry matter basis.

The pet food composition may be in the form of a supplement and/or an additive adapted to be incorporated into or fed with a pet food composition. The pet food composition in some instance may be formulated such that the medium chain triglyceride(s) having an octanoate component comprise about 50% or more of the total weight of the fat in the pet food composition. For example, the total amount of fat in the pet food composition may be comprised of medium chain triglyceride(s) having at least one aliphatic chain comprising a octanoate component in an amount of from about 50 up to 100 wt. %, about 55 up to 100 wt. %, about 60 up to 100 wt. %, about 65 up to 100 wt. %, about 70 up to 100 wt. %, about 75 up to 100 wt. %, about 80 up to 100 wt. %, about 85 up to 100 wt. %, about 90 up to 100 wt. %, about 95 up to 100 wt. %; from about 50 to about 95 wt. %, about 55 to about 95 wt. %, about 60 to about 95 wt. %, about 65 to about 95 wt. %, about 70 to about 95 wt. %, about 75 to about 95 wt. %, about 80 to about 95 wt. %, about 85 to about 95 wt. %, about 90 to about 95 wt. %; from about 50 to about 90 wt. %, about 55 to about 90 wt. %, about 60 to about 90 wt. %, about 65 to about 90 wt. %, about 70 to about 90 wt. %, about 75 to about 90 wt. %, about 80 to about 90 wt. %, about 85 to about 90 wt. %; from about 50 to about 85 wt. %, about 55 to about 85 wt. %, about 60 to about 85 wt. %, about 65 to about 85 wt. %, about 70 to about 85 wt. %, about 75 to about 85 wt. %, about 80 to about 85 wt. %; from about 50 to about 80 wt. %, about 55 to about 80 wt. %, about 60 to about 80 wt. %, about 65 to about 80 wt. %, about 70 to about 80 wt. %, about 75 to about 80 wt. %; from about 50 to about 75 wt. %, about 55 to about 75 wt. %, about 60 to about 75 wt. %, about 65 to about 75 wt. %, about 70 to about 75 wt. %; from about 50 to about 70 wt. %, about 55 to about 70 wt. %, about 60 to about 70 wt. %, about 65 to about 70 wt. %; from about 50 to about 65 wt. %, about 55 to about 65 wt. %, about 60 to about 65 wt. %; from about 50 to about 60 wt. %, about 55 to about 60 wt. %, based on the total weight of the fat in the pet food composition on a dry matter basis.

The total amount of one or more medium chain triglyceride(s) may consist essentially of or consists of medium chain triglyceride having an octanoate component. For example, the total amount of medium chain triglyceride may be comprised of about 80 to about 100 wt. %, about 85 to about 100 wt. %, about 90 to about 100 wt. %, about 95 to about 100 wt. %, of a medium chain triglyceride(s) having at least one aliphatic chain comprising a octanoate component, based on the total weight of medium chain triglyceride(s). In some embodiments, the fat of the pet food composition may be comprised of about 98 wt. %, about 99 wt. %, or about 100 wt. % of a medium chain triglyceride(s) having at least one aliphatic chain comprising a octanoate component, based on the total weight of medium chain triglyceride(s).

In addition to the at least one medium chain triglyceride having at least one aliphatic chain comprising an octanoate component, the fat may comprise one or more additional triglyceride(s). The additional triglyceride(s) may have one, two, or all three aliphatic chain components selected from pentanoic acid, hexanoic acid, heptanoic acid, octanoic acid, nonanoic acid, decanoic acid, undecanoic acid, dodecanoic acid, tridecanoic acid, tetradecanoic acid, pentadecanoic acid, hexadecanoic acid, heptadecanoic acid, octadecanoic acid, nonadecanoic acid, eicosanoic acid, and mixtures thereof. In at least one preferred embodiment, the additional triglyceride(s) is selected from medium chain triglycerides.

As used herein, “medium chain triglycerides” refers to a triglyceride having at least one aliphatic carbon chain comprised of 6 to 10 carbons. For example, a medium chain triglyceride may have one, two, or three aliphatic carbons chains of 6 to 10 carbons. For example, the pet food composition may comprise two, three, four, five, six, seven or more triglycerides, all of which may be selected from medium chain triglycerides. In some embodiments, the additional medium chain triglyceride has an aliphatic carbon chain of 6 carbons, 7 carbons, 8 carbons, 9 carbons, and/or 10 carbons. For example, the aliphatic carbon chain of the medium chain triglyceride may include from 6 to 9 carbons, 6 to 8 carbons, 6 or 7 carbons; 7 to 10 carbons, 7 to 9 carbons, 7 or 8 carbons; 8 to 10 carbons, or 8 or 9 carbons, or 9 or 10 carbons. In some embodiments, the medium chain triglyceride(s) comprises an aliphatic carbon chain having 6 carbons, 8 carbons, or 10 carbons. For example, the additional medium chain triglyceride may have an aliphatic carbon chain selected from caprylate, caprate, and/or decanoate.

The inventors recognized that medium chain triglycerides having an octanoate (C8) component is more ketogenic than medium chain triglycerides having a decanoate (C10) component. Without being limited to any particular theory, it is believed that pet food compositions having an enhanced amount of medium chain triglycerides having an octanoate component and, preferably a reduced amount of medium chain triglycerides having a deconoate component can yield metabolic benefits achieved under ketotsis. Additionally and/or alternatively, it was unexpectedly determined that medium chain triglycerides having an aliphatic chain of 10 to 12 carbons may reduce the pet food compositions ability to increase ketones (e.g. BHB).

In certain embodiments, the pet food compositions may be formulated to control the amount of medium chain triglycerides having an aliphatic chain of 10 to 12 carbons (e.g., a decanoate (C10) component). In at least one preferred embodiment, the fat of the pet food composition comprises a medium chain triglyceride having an aliphatic chain comprising an octanoate component and a medium chain triglyceride having an aliphatic chain comprising 10 to 12 carbons (e.g., a decanoate component). The medium chain triglyceride having an aliphatic chain comprising 10 to 12 carbons (e.g. a decanoate component) is preferably about 50 wt. % or less, about 45 wt. % or less, about 40 wt. % or less, about 35 wt. % or less, about 30 wt. % or less, about 25 wt. % or less, about 20 wt. % or less, or about 15 wt. % or less, of the fat of the pet food composition, based on total weight of the fat in the pet food composition on a dry matter basis. In certain embodiments, the pet food composition may be adapted such that the fat comprises medium chain triglycerides having 10 to 12 carbons (such as, a decanoate component) in an amount of about 4 wt. % or less, e.g., about 3 wt. % or less, about 2 wt. % or less, about 1 wt. % or less, about wt. % or less, about 0.1 wt. % or less, about 0.05 wt. % or less, about 0.02 wt. % or less, or about wt. % or less, based on the total weight of the fat in the pet food composition on a dry matter basis. In at least one embodiment, the pet food composition may be essentially free of or free of medium chain triglycerides having 10 to 12 carbons and/or a decanoate component.

Sources of medium chain triglyceride(s) include, for example, coconut oil, palm oil, and palm kernel oil. Coconut oil is an oil extracted from the kernel or meat of matured coconuts. Palm kernel oil is derived from the kernel of the oil palm, and palm oil is derived from the oil palm fruit itself. Coconut oil, palm oil, and palm kernel oil all comprise a high content of saturated fat. In certain embodiments, the one or more medium chain triglyceride(s) is manufactured by the esterification of glycerin and fatty acids that originate from at least one of coconut oil and palm kernel oil, such as Captex® 355 medium chain triglyceride(s).

Additionally or alternatively, the pet food composition may be formulated to have a weight ratio of the medium chain triglyceride having an aliphatic chain comprising an octanoate component to the medium chain triglyceride having an aliphatic chain comprising an decanoate component of from about 2:1 to about 20:1. In some embodiments, the weight ratio of the medium chain triglyceride(s) having an aliphatic chain comprising an octanoate component to the medium chain triglyceride(s) having an aliphatic chain comprising an decanoate component is from about 2:1 to about 20:1, about 2:1 to about 17:1, about 2:1 to about 15:1, about 2:1 to about 13:1, about 2:1 to about 11:1, about 2:1 to about 9:1, about 2:1 to about 7:1; from about 4:1 to about 20:1, about 4:1 to about 17:1, about 4:1 to about 15:1, about 4:1 to about 13:1, about 4:1 to about 11:1, about 4:1 to about 9:1, about 4:1 to about 7:1; from about 5:1 to about 20:1, about 5:1 to about 17:1, about 5:1 to about 15:1, about 5:1 to about 13:1, about 5:1 to about 11:1, about 5:1 to about 9:1, about 5:1 to about 7:1; from about 6:1 to about 20:1, about 6:1 to about 17:1, about 6:1 to about 15:1, about 6:1 to about 13:1, about 6:1 to about 11:1, about 6:1 to about 9:1, or any range or subrange thereof. In certain embodiments, the weight ratio of the medium chain triglyceride(s) having an aliphatic chain comprising an octanoate component to the medium chain triglyceride(s) having an aliphatic chain comprising a decanoate component is from about 250:1 to about 500:1, e.g., from about 300:1 to about 500:1, about 350:1 to about 500:1, about 400:1 to about 500:1; from about 250:1 to about 450:1, about 300:1 to about 450:1, about 350:1 to about 450:1, about 400:1 to about 450:1; from about 250:1 to about 400:1, about 300:1 to about 400:1, about 350:1 to about 400:1, or any range or subrange thereof.

In some embodiments, the pet food compositions are formulated such that the total amount of medium chain triglyceride(s) comprises about 7 wt. % or more of the fat in the pet food composition. For example, the fat may be comprised of medium chain triglyceride(s) in an amount of about 7 to about 60 wt. %, about 7 to about 50 wt. %, about 7 to about 45 wt. %, about 7 to about 40 wt. %, about 7 to about 37 wt. %, about 7 to about 34 wt. %, about 7 to about 31 wt. %, about 7 to about 28 wt. %, about 7 to about 25 wt. %, about 7 to about 22 wt. %, about 7 to about 17 wt. %, about 7 to about 12 wt. %; from about 12 to about 60 wt. %, about 12 to about 50 wt. %, about 12 to about 45 wt. %, about 12 to about 40 wt. %, about 12 to about 37 wt. %, about 12 to about 34 wt. %, about 12 to about 31 wt. %, about 12 to about 28 wt. %, about 12 to about 25 wt. %, about 12 to about 22 wt. %, about 12 to about 17 wt. %; from about 17 to about 60 wt. %, about 17 to about 50 wt. %, about 17 to about 45 wt. %, about 17 to about 40 wt. %, about 17 to about 37 wt. %, about 17 to about 34 wt. %, about 17 to about 31 wt. %, about 17 to about 28 wt. %, about 17 to about 25 wt. %; from about 23 to about 60 wt. %, about 23 to about 50 wt. %, about 23 to about 45 wt. %, about 23 to about 40 wt. %, about 23 to about 37 wt. %, about 23 to about 34 wt. %, about 23 to about 31 wt. %; from about 27 to about 60 wt. %, about 27 to about 50 wt. %, about 27 to about 45 wt. %, about 27 to about 40 wt. %, about 27 to about 37 wt. %, about 27 to about 34 wt. %, about 27 to about 31 wt. %; from about 31 to about 60 wt. %, about 31 to about 50 wt. %, about 31 to about 45 wt. %, about 31 to about 40 wt. %, about 31 to about 37 wt. %; from about 35 to about 60 wt. %, about 35 to about 50 wt. %, about 35 to about 45 wt. %, about 35 to about 40 wt. %; from about 39 to about 60 wt. %, about 39 to about 50 wt. %, about 39 to about 45 wt. %; from about 43 to about 60 wt. %, about 43 to about 50 wt. %, about 47 to about 60 wt. %, or any range or subrange thereof, based on the total weight of the fat in the pet food composition on a dry matter basis.

The pet food composition may be in the form of a supplement and/or an additive adapted to be incorporated into or fed with a pet food composition. The pet food composition in some instance may have a total amount of fat in the pet food composition may be comprised of medium chain triglyceride(s) in an amount of from about 50 up to 100 wt. %, about 55 up to 100 wt. %, about 60 up to 100 wt. %, about 65 up to 100 wt. %, about 70 up to 100 wt. %, about 75 up to 100 wt. %, about 80 up to 100 wt. %, about 85 up to 100 wt. %, about 90 up to 100 wt. %, about 95 up to 100 wt. %; from about 50 to about 95 wt. %, about 55 to about 95 wt. %, about 60 to about 95 wt. %, about 65 to about 95 wt. %, about 70 to about 95 wt. %, about 75 to about 95 wt. %, about 80 to about 95 wt. %, about 85 to about 95 wt. %, about 90 to about 95 wt. %; from about 50 to about 90 wt. %, about 55 to about 90 wt. %, about 60 to about 90 wt. %, about 65 to about 90 wt. %, about 70 to about 90 wt. %, about 75 to about 90 wt. %, about 80 to about 90 wt. %, about 85 to about 90 wt. %; from about 50 to about 85 wt. %, about 55 to about 85 wt. %, about 60 to about 85 wt. %, about 65 to about 85 wt. %, about 70 to about 85 wt. %, about 75 to about 85 wt. %, about 80 to about 85 wt. %; from about 50 to about 80 wt. %, about 55 to about 80 wt. %, about 60 to about 80 wt. %, about 65 to about 80 wt. %, about 70 to about 80 wt. %, about 75 to about 80 wt. %; from about 50 to about 75 wt. %, about 55 to about 75 wt. %, about 60 to about 75 wt. %, about 65 to about 75 wt. %, or about 70 to about 75 wt. %, based on the total weight of the fat in the pet food composition on a dry matter basis.

Additionally or alternatively, the one or more triglyceride(s) may include a polyunsaturated fatty acid component selected from omega-3 fatty acids, omega-6 fatty acids, or a combination of two or more thereof. Examples of omega-3 fatty acid components that may comprise the aliphatic chain of the additional triglyceride include those selected from linolenic acid, stearidonic acid, eicosatetraenoic acid, eicosapentaenoic acid, docosapentaenoic acid, and/or docosahexaenoic acid. Examples of omega-6 fatty acid components that may comprise the aliphatic chain of an additional triglyceride include linolenic acid, calendic acid, eicosadienoic acid, arachidonic acid, docosadienoic acid, adrenic acid, osbond acid, tetracosatetraenoic acid, and/or tetracosapentaenoic acid. In some embodiments, the polyunsaturated fatty acid component of the additional triglyceride is selected from linolenic acid, and/or arachidonic acid.

The pet food compositions may, optionally, include one or more fatty acid(s). The fatty acid(s) when present in the pet food composition is typically in an amount from about 6 to about 65 wt. %, based on the total weight of the pet food composition on a dry matter basis. For example, the fatty acid may present in an amount from about 6 to about 65 wt. %, about 6 to about 60 wt. %, about 6 to about 55 wt. %, about 6 to about 50 wt. %, about 6 to about 45 wt. %, about 6 to about 40 wt. %, about 6 to about 35 wt. %, about 6 to about 30 wt. %, about 6 to about 25 wt. %, about 6 to about 20 wt. %, about 6 to about 15 wt. %; from about 15 to about 65 wt. %, about 15 to about 60 wt. %, about 15 to about 55 wt. %, about 15 to about 50 wt. %, about 15 to about 45 wt. %, about 15 to about 40 wt. %, about 15 to about 35 wt. %, about 15 to about 30 wt. %, about 15 to about 25 wt. %, about 15 to about 20 wt. %, about 15 to about 15 wt. %; from about 25 to about 65 wt. %, about 25 to about 60 wt. %, about 25 to about 55 wt. %, about 25 to about 50 wt. %, about 25 to about 45 wt. %, about 25 to about 40 wt. %, about 25 to about 35 wt. %, about 25 to about 30 wt. %, about 25 to about 25 wt. %, about 25 to about 20 wt. %, about 25 to about 15 wt. %; from about 35 to about 65 wt. %, about 35 to about 60 wt. %, about 35 to about 55 wt. %, about 35 to about 50 wt. %, about 35 to about 45 wt. %; from about 45 to about 65 wt. %, about 45 to about 60 wt. %, or about 45 to about 55 wt. %, including ranges and subranges thereof, based on the total weight of the pet food composition.

The one or more fatty acid(s) may be selected from omega-3 fatty acids, omega-6 fatty acids, lauric acid, myristic acid, palmitic acid, palmitoleic acid, margaric acid, margaroleic acid, stearic acid, oleic acid, stearidonic acid, gadoleic acid, behenic acid, erucic acid, docosatetra acid, and a combination of two or more thereof. In at least one preferred embodiment, the one or more fatty acid comprises an omega-3 fatty acid, an omega-6 fatty acid, or a combination of two or more thereof. Examples of omega-3 fatty acids include linolenic acid, stearidonic acid, eicosatetraenoic acid, eicosapentaenoic acid, docosapentaenoic acid, and docosahexaenoic acid. Examples of omega-6 fatty acids include linoleic acid, calendic acid, eicosadienoic acid, arachidonic acid, docosadienoic acid, adrenic acid, osbond acid, tetracosatetraenoic acid, and tetracosapentaenoic acid.

The fat may comprise about 1 to about 20 wt. % of omega-3 fatty acid, based on the total weight of the fat. For instance, the fat may be comprised of about 1 to about 16 wt. %, about 1 to about 14 wt. %, about 1 to about 12 wt. %, about 1 to about 10 wt. %, about 1 to about 8 wt. %, about 1 to about 6 wt. %, about 1 to about 5 wt. %; from about 3 to about 20 wt. %, about 3 to about 16 wt. %, about 3 to about 14 wt. %, about 3 to about 12 wt. %, about 3 to about 10 wt. %, about 3 to about 8 wt. %, about 3 to about 6 wt. %, about 3 to about 5 wt. %; from about 5 to about 20 wt. %, about 5 to about 16 wt. %, about 5 to about 14 wt. %, about 5 to about 12 wt. %, about 5 to about 10 wt. %, about 5 to about 8 wt. %; from about 7 to about 20 wt. %, about 7 to about 16 wt. %, about 7 to about 14 wt. %, about 7 to about 12 wt. %, or about 7 to about 10 wt. %, including ranges and subranges thereof, based on the total weight of the fat.

The fat may comprise about 5 to about 40 wt. % of omega-6 fatty acid, based on the total weight of the fat. For instance, the fat may be comprised of about 5 to about 40 wt. %, about 5 to about 35 wt. %, about 5 to about 30 wt. %, about 5 to about 25 wt. %, about 5 to about 20 wt. %, about 5 to about 15 wt. %; from about 10 to about 40 wt. %, about 10 to about 35 wt. %, about 10 to about 30 wt. %, about 10 to about 25 wt. %, about 10 to about 20 wt. %, about 10 to about 15 wt. %; from about 15 to about 40 wt. %, about 15 to about 35 wt. %, about 15 to about 30 wt. %, about 15 to about 25 wt. %; from about 20 to about 40 wt. %, about 20 to about 35 wt. %, about 20 to about 30 wt. %; from about 25 to about 40 wt. %, about 25 to about 35 wt. %, or about 30 to about 40 wt. %, including ranges and subranges thereof, based on the total weight of the fat.

The pet food composition, in some embodiments, comprises one or more omega-3 fatty acid and one or more omega-6 fatty acid. In some instance, the pet food composition is formulated to have a weight ratio of omega-3 fatty acid(s) to omega-6 fatty acid(s) of from about 5:1 to about 1:20, about 4:1 to about 1:20, about 3:1 to about 1:20, about 2:1 to about 1:20, about 1:1 to about 1:20; about 5:1 to about 1:20; from about 1:1 to about 1:20, about 1:1 to about 1:18, about 1:1 to about 1:16, about 1:1 to about 1:14, about 1:1 to about 1:12, about 1:1 to about 1:10, about 1:1 to about 1:8, about 1:1 to about 1:6, about 1:1 to about 1:5, about 1:1 to about 1:4, about 1:1 to about 1:3, or about 1:1 to about 1:2, including any ranges or subranges thereof.

The fat may be incorporated completely within the food composition, deposited on the outside of the pet food composition, or a mixture of the two methods. In some embodiments, the pet food compositions further include an effective amount of one or more substances selected from the group consisting of glucosamine, chondroitin, chondroitin sulfate, methylsulfonylmethane (“MSM”), creatine, antioxidants, Perna canaliculata, and mixtures thereof.

Fat can be supplied by any of a variety of sources known by those skilled in the art, including meat, meat by-products, canola oil, fish oil such as anchovy oil and menhaden oil, and plants. Meat fat sources include poultry fat, turkey fat, pork fat, lard, tallow, and beef fat. Plant fat sources include wheat, flaxseed, rye, barley, rice, sorghum, corn, oats, millet, wheat germ, corn germ, soybeans, peanuts, and cottonseed, as well as oils derived from these and other plant fat sources such as corn oil, soybean oil, cottonseed oil, palm oil, palm kernel oil, linseed oil, canola oil, rapeseed oil, and/or olestra.

In some cases, the fat in the pet food composition is a crude fat. Crude fat may be included into the compositions in the amounts disclosed above with respect of the total fat, such as from about 8 to about 50 wt. %, based on the total weight of the pet food composition on a dry matter basis. In some embodiments, the pet food composition comprises crude fat in an amount of about 10 to about 40 wt. %, about 12 to about 35 wt. %, about 14 to about 30 wt. %, about 16 to about 24 wt. %, based on the total weight of the pet food composition on a dry matter basis. In some cases, it may be preferable that about 50 wt. % or more, about 60 wt. % or more, about 70 wt. % or more, about 80 wt. % or more, or about 90 wt. % or more of the total fat is obtained from an animal source. Alternatively, about 50 wt. % or more, about 60 wt. % or more, about 70 wt. % or more, about 80 wt. % or more, or about 90 wt. % or more of the total fat may be obtained from a plant source.

The pet food composition includes protein in an amount that may vary. For instance, the pet food composition may include protein in an amount from about 40 g/1000 kcal to about 150 g/1000 kcal, based on the total metabolizable energy of the pet food composition on a dry matter basis. For example, the amount of protein in the pet food composition may be from about 40 g/1000 kcal to about 130 g/1000 kcal, about 40 g/1000 kcal to about 110 g/1000 kcal, about 40 g/1000 kcal to about 100 g/1000 kcal, about 40 g/1000 kcal to about 90 g/1000 kcal, about 40 g/1000 kcal to about 80 g/1000 kcal, about 40 g/1000 kcal to about 70 g/1000 kcal, about 40 g/1000 kcal to about 60 g/1000 kcal; from about 60 g/1000 kcal to about 150 g/1000 kcal, about 60 g/1000 kcal to about 130 g/1000 kcal, about 60 g/1000 kcal to about 110 g/1000 kcal, about 60 g/1000 kcal to about 100 g/1000 kcal, about 60 g/1000 kcal to about 90 g/1000 kcal, about 60 g/1000 kcal to about 80 g/1000 kcal; from about 80 g/1000 kcal to about 150 g/1000 kcal, about 80 g/1000 kcal to about 130 g/1000 kcal, about 80 g/1000 kcal to about 110 g/1000 kcal, about 80 g/1000 kcal to about 100 g/1000 kcal; from about 100 g/1000 kcal to about 150 g/1000 kcal, about 100 g/1000 kcal to about 130 g/1000 kcal; from about 120 g/1000 kcal to about 150 g/1000 kcal, or about 130 g/1000 kcal to about 150 g/1000 kcal, including ranges and subranges thereof, based on the total metabolizable energy of the pet food composition on a dry matter basis.

The protein of the pet food composition may comprises one or more amino acids selected from Tryptophan, Taurine, Histidine, Carnitine, Carnosine, Alanine, Cysteine, Arginine, Methionine (including DL-methionine, and L-methionine), Tryptophan, Lysine, Asparagine, Aspartate (Aspartic acid), Phenylalanine, Valine, Threonine, Isoleucine, Histidine, Leucine, Glycine, Glutamine, Taurine, Tyrosine, Homocysteine, Ornithine, Citruline, Glutamate (Glutamic acid), Proline, and/or Serine, and a combination of two or more thereof. In some cases, the one or more amino acid(s) may comprise essential amino acids. Essential amino acids are amino acids that cannot be synthesized de novo, or in sufficient quantities by an organism and thus must be supplied in the diet. Essential amino acids vary from species to species, depending upon the organism's metabolism. For example, it is generally understood that the essential amino acids for dogs and cats (and humans) are phenylalanine, leucine, methionine, lysine, isoleucine, valine, threonine, tryptophan, histidine and arginine. In addition, taurine, while technically not an amino acid but a derivative of cysteine, is an essential nutrient for cats.

A portion of the protein in the composition may be digested (e.g., hydrolyzed) protein. For example, pet food composition may be formulated to a ratio of digested (e.g., hydrolyzed) protein to non-digestible protein from about 4:6 to about 10:1, about 4:5 to about 10:1, about 1:1 to about 10:1; from about 1:1 to about 8:1, about 1:1 to about 7:1, about 1:1 to about 6:1, about 1:1 to about about 1:1 to about 4:1, about 1:1 to about 3:1; from about 3:1 to about 10:1, about 3:1 to 5:1, about 9:1, about 3:1 to about 8:1, about 3:1 to about 7:1, about 3:1 to about 6:1, about 3:1 to about 5:1; from about 5:1 to about 10:1, about 5:1 to about 9:1, about 5:1 to about 8:1, about 5:1 to about 7:1, or any range or subrange thereof.

Protein may be supplied by any of a variety of sources known by those of ordinary skill in the art including plant sources, animal sources, microbial sources or a combination of these. For example, animal sources may include meat, meat-by products, seafood, dairy, eggs, etc. Meats, for example, may include animal flesh such as poultry, fish, and mammals including cattle, pigs, sheep, goats, and the like. Meat by-products may include, for example, lungs, kidneys, brain, livers, stomachs and intestines. Plant protein includes, for example, soybean, cottonseed, and peanuts. Microbial sources may be used to synthesize amino acids (e.g., lysine, threonine, tryptophan, methionine) or intact protein such as protein from sources listed below.

Examples of protein or protein ingredients may comprise chicken meals, chicken, chicken by-product meals, lamb, lamb meals, turkey, turkey meals, beef, beef by-products, viscera, fish meal, enterals, kangaroo, white fish, venison, soybean meal, soy protein isolate, soy protein concentrate, corn gluten meal, corn protein concentrate, distillers dried grains, and/or distillers dried grain solubles and single-cell proteins, for example yeast, algae, and/or bacteria cultures.

The protein can be intact, completely hydrolyzed, or partially hydrolyzed. The protein content of foods may be determined by any number of methods known by those of skill in the art, for example, as published by the Association of Official Analytical Chemists in Official Methods of Analysis (“OMA”), method 988.05. The amount of protein in a composition disclosed herein may be determined based on the amount of nitrogen in the composition according to methods familiar to one of skill in the art.

The pet food compositions are typically formulated to include fiber in an amount from about 5 g/1000 kcal to about 55 g/1000 kcal of a fiber, based on the total metabolizable energy of the pet food composition on a dry matter basis. For example, the amount of fiber in the pet food composition may be from about 5 g/1000 kcal to about 45 g/1000 kcal, about 5 g/1000 kcal to about 35 g/1000 kcal, about 5 g/1000 kcal to about 25 g/1000 kcal, about 5 g/1000 kcal to about 15 g/1000 kcal; from about 10 g/1000 kcal to about 55 g/1000 kcal, about 10 g/1000 kcal to about 45 g/1000 kcal, about 10 g/1000 kcal to about 35 g/1000 kcal, about 10 g/1000 kcal to about 25 g/1000 kcal, about 10 g/1000 kcal to about 15 g/1000 kcal; about 15 g/1000 kcal to about 55 g/1000 kcal, about 15 g/1000 kcal to about 45 g/1000 kcal, about 15 g/1000 kcal to about 35 g/1000 kcal, about 15 g/1000 kcal to about 25 g/1000 kcal; from about 25 g/1000 kcal to about 55 g/1000 kcal, about 25 g/1000 kcal to about 45 g/1000 kcal, about 25 g/1000 kcal to about 35 g/1000 kcal; from about 35 g/1000 kcal to about 55 g/1000 kcal, about 35 g/1000 kcal to about 45 g/1000 kcal; or about 45 g/1000 kcal to about 55 g/1000 kcal, including ranges and subranges thereof, based on the total metabolizable energy of the pet food composition on a dry matter basis.

The total amount of fiber present in the pet food composition generally comprises an amount of crude fiber and dietary fiber. Crude fiber includes indigestible components contained in cell walls and cell contents of plants such as grains, e.g., hulls of grains such as rice, corn, and beans. Dietary fiber refers to components of a plant that are resistant to digestion by an animal's digestive enzymes. Dietary fiber includes soluble fiber and insoluble fiber. Soluble fibers are resistant to digestion and absorption in the small intestine and undergo complete or partial fermentation in the large intestine, e.g., beet pulp, guar gum, chicory root, psyllium, pectin, blueberry, cranberry, squash, apples, oats, beans, citrus, barley, or peas. Insoluble fibers can be supplied by any of a variety of sources, including, for example, cellulose, whole-wheat products, wheat oat, corn bran, flax seed, grapes, celery, green beans, cauliflower, potato skins, fruit skins (e.g., pear skin), vegetable skins, peanut hulls, almond shell, walnut shell, pecan shell, citrus pulp, beet pulp, and soy fiber. In some embodiments, the dietary fiber may be chosen from pecan shell, citrus pulp, beet pulp, and a combination of two or more thereof. Crude fiber includes indigestible components contained in cell walls and cell contents of plants such as grains, for example, hulls of grains such as rice, corn, and beans.

The pet food compositions may include one or more sclereid. The one or more sclereids may be obtained from nut shells, such as pecan shells, almonds shells, walnut shells, etc.; from seeds, such as pea, bean, soybean seeds, etc.; and/or from fruit skins, such as pear skin, etc. In one embodiment, the pet food composition includes pecan shell particles comprising one or more sclereids. The sclereid(s) may have a microscopic structure and, preferably, serve as an abrasive. For instance, the sclereid(s) may be selected to provide mechanical disruption of debris, food, and/or bacteria on the teeth of a pet consuming a pet food composition disclosed herein, which contains such sclereid(s). In at least one embodiment, the sclereid(s) may be selected to include one or more polyphenols.

In some embodiments, the pet food composition has a weight ratio of insoluble fiber to soluble fiber from about 20:1 to about 8:1. For example, the pet food composition may have a weight ratio of insoluble fiber to soluble fiber from about 18:1 to about 8:1, about 16:1 to about 8:1, about 14:1 to about 8:1, about 12:1 to about 8:1, about 10:1 to about 8:1. The pet food composition may have a weight ratio of insoluble fiber to soluble fiber of about 11:1.

Additionally and/or alternatively, the fiber component of the pet food composition may comprise an acid detergent fiber and/or a neutral detergent fiber. In some embodiments, the pet food composition has a weight ratio of the acid detergent fiber to the neutral detergent fiber of from about 3:1 to about 1:3. Preferably, the weight ratio of the acid detergent fiber to the neutral detergent fiber is from about 2:1 to about 1:2. In some instances, the weight ratio of the acid detergent fiber to the neutral detergent fiber is from about 1.75:1 to about 1:1.75, about 1.5:1 to about 1:1.5, about 1.25:1 to about 1:1.25. In at least one embodiment, the weight ratio of the acid detergent fiber to the neutral detergent fiber is about 1:1.

The pet food composition may further comprise ash. As described herein, ash consists of compounds that are not organic or water, generally produced by combustion of biological materials. The ash may be present in the pet food composition in an amount ranging from about 1 to about 10 wt. %, based on the total weight of the food composition on a dry weight basis, including all amounts and sub-ranges there-between. In some embodiment, the ash may be present in the food composition in an amount ranging from about 1 to about 8 wt. %, about 1 to about 6 wt. %, about 1 to about 5 wt. %, about 1 to about 4 wt. %, about 1 to about 3 wt. %, about 1 to about 2 wt. %; from about 2 to about 10 wt. %, about 2 to about 8 wt. %, about 2 to about 6 wt. %, about 2 to about 5 wt. %, about 2 to about 4 wt. %; from about 3 to about 10 wt. %, about 3 to about 8 wt. %, about 3 to about 6 wt. %, about 3 to about 5 wt. %; from about 4 to about 10 wt. %, about 4 to about 8 wt. %, about 4 to about 6 wt. %; from about 5 to about 10 wt. %, about 5 to about 8 wt. %, or any range or subrange thereof, based on the total weight of the pet food composition on a dry weight basis.

The pet food composition preferably contains about 3 g/1000 kcal to about 130 g/1000 kcal of carbohydrates, based on the total metabolizable energy of the pet food composition on a dry matter basis. In some embodiments, the pet food compositions includes carbohydrates in an amount from 3 g/1000 kcal to about 100 g/1000 kcal, 3 g/1000 kcal to about 70 g/1000 kcal, 3 g/1000 kcal to about 50 g/1000 kcal, 3 g/1000 kcal to about 30 g/1000 kcal, 3 g/1000 kcal to about 20 g/1000 kcal, 3 g/1000 kcal to about 10 g/1000 kcal; from about 10 g/1000 kcal to about 130 g/1000 kcal, 10 g/1000 kcal to about 100 g/1000 kcal, 10 g/1000 kcal to about 70 g/1000 kcal, 10 g/1000 kcal to about 50 g/1000 kcal, 10 g/1000 kcal to about 30 g/1000 kcal, 10 g/1000 kcal to about 20 g/1000 kcal; from about 15 g/1000 kcal to about 130 g/1000 kcal, 15 g/1000 kcal to about 100 g/1000 kcal, 15 g/1000 kcal to about 70 g/1000 kcal, 15 g/1000 kcal to about 50 g/1000 kcal, 15 g/1000 kcal to about 30 g/1000 kcal; from about 20 g/1000 kcal to about 130 g/1000 kcal, 20 g/1000 kcal to about 100 g/1000 kcal, 20 g/1000 kcal to about 70 g/1000 kcal, 20 g/1000 kcal to about 50 g/1000 kcal, 20 g/1000 kcal to about 30 g/1000 kcal; from about 30 g/1000 kcal to about 130 g/1000 kcal, 30 g/1000 kcal to about 100 g/1000 kcal, 30 g/1000 kcal to about 70 g/1000 kcal, 30 g/1000 kcal to about 50 g/1000 kcal; from about 40 g/1000 kcal to about 130 g/1000 kcal, about 40 g/1000 kcal to about 110 g/1000 kcal, about 40 g/1000 kcal to about 100 g/1000 kcal, about 40 g/1000 kcal to about 90 g/1000 kcal, about 40 g/1000 kcal to about 80 g/1000 kcal, about 40 g/1000 kcal to about 70 g/1000 kcal, about 40 g/1000 kcal to about 60 g/1000 kcal; from about 60 g/1000 kcal to about 130 g/1000 kcal, about 60 g/1000 kcal to about 110 g/1000 kcal, about 60 g/1000 kcal to about 100 g/1000 kcal, about 60 g/1000 kcal to about 90 g/1000 kcal, about 60 g/1000 kcal to about 80 g/1000 kcal; from about 80 g/1000 kcal to about 130 g/1000 kcal, about 80 g/1000 kcal to about 110 g/1000 kcal, about 80 g/1000 kcal to about 100 g/1000 kcal; or about 100 g/1000 kcal to about 130 g/1000 kcal, including ranges and subranges thereof, based on the total metabolizable energy of the pet food composition on a dry matter basis.

The term “carbohydrate” as used herein includes polysaccharides (e.g., starches and dextrins) and sugars (e.g., sucrose, lactose, maltose, glucose, and fructose) that are metabolized for energy when hydrolyzed. Generally, carbohydrate percentage can be calculated as nitrogen free extract (“NFE”), which can be calculated as follows: NFE %=100%−(moisture %)−(protein %)−(fat %)−(ash %)−(crude fiber %). One skilled in the art could manipulate the texture of the final product by properly balancing carbohydrate sources. For example, short chain polysaccharides lend to be sticky and gluey, and longer chain polysaccharides are less sticky and gluey than the shorter chain; the desired texture of this hybrid food is achieved by longer chain polysaccharide and modified starches such as native or modified starches, cellulose and the like. The carbohydrate mixture may additionally comprise optional components such as added salt, spices, seasonings, vitamins, minerals, flavorants, colorants, and the like. The amount of the optional components is at least partially dependent on the nutritional requirements for different life stages of animals.

Carbohydrates can be supplied by any of a variety of sources known by those skilled in the art, including, but not limited to, oat fiber, cellulose, peanut hulls, beet pulp, parboiled rice, cornstarch, corn gluten meal, cereal, and sorghum. Grains supplying carbohydrates can include, but are not limited to, wheat, durum, semolina, corn, barley, and rice. In certain embodiments, the carbohydrate component comprises a mixture of one or more carbohydrate sources. Carbohydrates content of foods can be determined by any number of methods known by those of skill in the art.

In certain embodiments, the pet food composition comprises moisture. The moisture may be present at various amounts or concentrations. In one embodiment, moisture may be present in an amount of from about 3 to about 20 wt. %, based on the total weight of the pet food composition. For example, moisture may be present in an amount of about 3 wt. %, about 5 wt. %, about 5.5 wt. %, about 6 wt. %, about 6.5 wt. %, about 7 wt. %, about 7.5 wt. %, about 8 wt. %, about 8.5 wt. %, about 9 wt. %, about 9.5 wt. %, about 10 wt. %, about 10.5 wt. %, about 11 wt. %, about 11.5 wt. %, about 12 wt. %, about 12.5 wt. %, about 13 wt. %, about 13.5 wt. %, about 14 wt. %, about 14.5 wt. %, or about 15 wt. %, based on the total weight of the pet food composition. In another example, moisture may be present in an amount of from about 6% to about 12%, about 9% to about 13%, about 9% to about 11%, or about 9% to about 13%, based on the total weight of the pet food composition. In certain embodiments, moisture is present in an amount of about 5% to about 12%, about 6% to about 11%, or about 7% to about 10.0%, based on the total weight of the pet food composition. In further embodiments, moisture is present in an amount of about 65% to about 85%, about 60% to about 80%, or about 60% to about 75%, based on the total weight of the pet food composition.

The pet food compositions may include one or more ingredients and/or sources of glucose mimetic, carotenoids, and/or arginine and derivatives thereof. Sources of glucose mimetics may comprise glucose anti-metabolites including 2-deoxy-D-glucose, 5-thio-D-glucose, 3-O-methylglucose, anhydrosugars including 1,5-anhydro-D-glucitol, 2,5-anhydro-D-glucitol, and 2,5-anhydro-D-mannitol, mannoheptulose, and/or avocado extract comprising mannoheptulose. Sources of carotenoids may include lutein, astaxanthin, zeaxanthin, bixin, lycopene, and/or beta-carotene. Sources of antioxidant ingredients may comprise tocopherols (vitamin E), vitamin C, vitamin A, plant-derived materials, carotenoids (described above), selenium, and/or CoQ10 (Co-enzyme Q10). The arginine present in the composition may be L-arginine, D-arginine, or a mixture thereof. The glucose mimetic(s), carotenoid(s), arginine(s), or a derivative(s) thereof may be included in the pet food composition in an amount from about 0.01 to about 10.0 wt. %, about 0.01 to about 5.0 wt. %, about 0.01 to about 2.0 wt. %, about 0.1 to about 10.0 wt. %, about 0.1 to about 5.0 wt. %, about 0.1 to about 2.0 wt. %; about 0.5 to about 5.0 wt. %, about 0.5 to about 2.0 wt. %, about 1.5 to about 5.0 wt. %, about 1.5 to about 2.0 wt. %, about 0.5 wt. %, about 1.0 wt. %, about 1.4 weight %, about 1.44 wt. %, about 1.8 wt. %, or about 2.0 wt. %, based on the total weight of the composition on a dry matter basis.

The pet food compositions disclosed herein may be wet or dry compositions, and the ingredients can be either incorporated into the food composition and/or on the surface of any composition component, such as, for example, by spraying, agglomerating, dusting, or precipitating on the surface. Additionally, the pet food compositions may be formulated and produced to be in various forms and/or consistencies. For instance, the pet food compositions may, for example, be a dry, moist or semi-moist animal food composition. “Semi-moist” refers to a food composition containing from about 25 to about 35 wt. % of water, based on the total weight of the pet food composition. “Moist” food refers to a food composition that has a water content of about 60 to 90 wt. % or greater, based on the total weight of the pet food composition. “Dry” food refers to a food composition with about 3 to about 12 wt. %, based on the total weight of the pet food composition, of water content and is often manufactured in the form of small bits or kibbles.

The food products may also include components of more than one consistency, for example, soft, chewy meat-like particles or pieces as well as kibble having an outer coating and an inner “core” component. In some embodiments, the pet food compositions may be in the form of a kibble or food kibble. As used herein, the term “kibble” or “food kibble” refers to a particulate pellet, e.g., like a component of feline or canine feeds. In some embodiments, a food kibble has a moisture, or water, content of less than 15% by weight, based on the total weight of the pet food composition. Food kibbles may range in texture from hard to soft. Food kibbles may range in internal structure from expanded to dense. Food kibbles may be formed by an extrusion process or a baking process. In non-limiting examples, a food kibble may have a uniform internal structure or a varied internal structure. For example, a food kibble may include a core and a coating to form a coated kibble. It should be understood that when the term “kibble” or “food kibble” is used, it can refer to an uncoated kibble or a coated kibble.

The pet food compositions of the present disclosure can additionally comprise other additives in amounts and combinations familiar to one of skill in the art. Such additives should be present in amounts that do not impair the purpose and effect provided by the invention. Examples of additives include substances with a stabilizing effect, organoleptic substances, processing aids, and substances that provide nutritional benefits.

Stabilizing substances may include, by way of example, substances that tend to increase the shelf life of the pet food composition. Other examples of other such additives potentially suitable for inclusion in the compositions of the invention include, for example, preservatives, antioxidants, synergists and sequestrants, packaging gases, stabilizers, emulsifiers, thickeners, gelling agents, and humectants. Examples of emulsifiers and/or thickening agents include gelatin, cellulose ethers, starch, starch esters, starch ethers, and modified starches. Additives for coloring, palatability, and nutritional purposes can include colorants, salts (including, but not limited to, sodium chloride, potassium citrate, potassium chloride, and other edible salts), vitamins (e.g., Vitamin B, Vitamin E, Vitamin C, etc.), minerals, and flavoring. Other additives can include glucosamine, chondroitin sulfate, vegetable extracts, herbal extracts, etc.

The concentration of such additives in the pet food composition may be up to about 5 wt. %, based on the total weight of the pet food composition on a dry matter basis. For example, the additives may be present in an amount from about 0.01 to about 5 wt. %, about 0.01 to about 4 wt. %, about 0.01 to about 4 wt. %, about 0.01 to about 3 wt. %, about 0.01 to about 2 wt. %, about to about 1 wt. %; about 0.1 to about 5 wt. %, about 0.1 to about 4 wt. %, about 0.1 to about 4 wt. %, about 0.1 to about 3 wt. %, about 0.1 to about 2 wt. %, about 0.1 to about 1 wt. %; about 1 to about 5 wt. %, about 1 to about 4 wt. %, about 1 to about 4 wt. %, about 1 to about 3 wt. %, about 1 to about 2 wt. %; about 2 to about 5 wt. %, about 2 to about 4 wt. %, about 2 to about 4 wt. %, about 2 to about 3 wt. %; about 3 to about 5 wt. %, about 3 to about 4 wt. %; or about 4 to about 5 wt. %, based on the total weight of the pet food composition on a dry matter basis. In some embodiments, the concentration of such additives (particularly where such additives are primarily nutritional balancing agents, such as vitamins and minerals) is from about 0 to about 2.0% by weight, based on the total weight of the pet food composition on a dry matter basis. In some embodiments, the concentration of such additives (again, particularly where such additives are primarily nutritional balancing agents) is from about 0 to about 1.0% by weight, based on the total weight of the pet food composition on a dry matter basis. Although the list of foregoing additives may be potentially suitable in some embodiments, one or more of the foregoing additives may be excluded from other embodiments of the pet food composition.

In specific embodiments, the pet food compositions and food products are formulated to address specific nutritional differences between species and breeds of animals, as well as one of more of the attributes of the animal. For example, cat foods, for example, are typically formulated based upon the life stage, age, size, weight, body composition, and breed.

Sources of proteins, carbohydrates, fats, vitamins, minerals, balancing agents, and the like, suitable for inclusion in the pet food compositions, and particularly in the food products to be administered in methods provided herein, may be selected from among those conventional materials known to those of ordinary skill in the art.

The pet food compositions may include, or in some instance exclude, one or more ingredients selected from beef broth, brewers dried yeast, egg, egg product, flax meal, DL methionine, amino acids, leucine, lysine, arginine, cysteine, cystine, aspartic acid, polyphosphates, sodium pyrophosphate, sodium tripolyphosphate; zinc chloride, copper gluconate, stannous chloride, stannous fluoride, sodium fluoride, triclosan, glucosamine hydrochloride, chondroitin sulfate, green lipped mussel, blue lipped mussel, methyl sulfonyl methane (MSM), boron, boric acid, phytoestrogens, phytoandrogens, genistein, diadzein, Lcarnitine, chromium picolinate, chromium tripicolinate, chromium nicotinate, acid/base modifiers, potassium citrate, potassium chloride, calcium carbonate, calcium chloride, sodium bisulfate; eucalyptus, lavender, peppermint, plasticizers, colorants, flavorants, sweeteners, buffering agents, slip aids, carriers, pH adjusting agents, natural ingredients, stabilizers, biological additives such as enzymes (including proteases and lipases), chemical additives, coolants, chelants, denaturants, drug astringents, emulsifiers, external analgesics, fragrance compounds, humectants, opacifying agents (such as zinc oxide and titanium dioxide), antifoaming agents (such as silicone), preservatives (such as butylated hydroxytoluene (BHT) and butylated hydroxyanisole (BHA), propyl gallate, benzalkonium chloride, EDTA, benzyl alcohol, potassium sorbate, parabens and mixtures thereof), reducing agents, solvents, hydrotropes, solubilizing agents, suspending agents (non-surfactant), solvents, viscosity increasing agents (aqueous and non-aqueous), sequestrants, keratolytics, egg shell membrane, ancient grain, and a combination of two or more thereof.

The pet food composition may be produced by various methods to achieve the desired pet food composition or desired form for the pet food composition. For example, dry food may be baked or extruded, then cut into individual shaped portions, such as kibbles. In some embodiments, the pet food composition may be prepared in a canned or wet form using conventional food preparation processes known to those of ordinary skill in the art. Typically, ground animal proteinaceous tissues are mixed with the other ingredients, such as cereal grains, suitable carbohydrate sources, fats, oils, and balancing ingredients, including special purpose additives such as vitamin and mineral mixtures, inorganic salts, cellulose, beet pulp and the like, and water in an amount sufficient for processing. The ingredients are mixed in a vessel suitable for heating while blending the components. Heating the mixture is carried out using any suitable manner, for example, direct steam injection or using a vessel fitted with a heat exchanger. Following addition of all of the ingredients of the formulation, the mixture may be heated to a temperature of from 50° F. to 212° F. Although temperatures outside this range can be used, they may be commercially-impractical without the use of other processing aids. When heated to the appropriate temperature, the material will typically be in the form of thick liquid, which is dispensed into cans. A lid is applied and the container is hermetically sealed. The sealed can is then placed in convention equipment designed for sterilization of the contents. Sterilization is usually accomplished by heating to temperatures of greater than 230° C. for an appropriate time depending on the temperature used, the nature of the composition, and related factors. The pet food compositions and food products of the present disclosure can also be added to or combined with food compositions before, during, or after their preparation.

In some embodiments, the food products may be prepared in a dry form using convention processes known to those of ordinary skill in the art. Typically, dry ingredients, including dried animal protein, plant protein, grains and the like are ground and mixed together. Liquid or moist ingredients, including fats, oils water, animal protein, water, and the like are added combined with the dry materials. The specific formulation, order of addition, combination, and methods and equipment used to combine the various ingredients can be selected from those known in the art. For example, in certain embodiments, the resulting mixture is process into kibbles or similar dry pieces, which are formed using an extrusion process in which the mixture of dry and wet ingredients is subjected to mechanical work at high pressure and temperature, forced through small openings or apertures, and cut off into the kibbles, e.g., with a rotating knife. The resulting kibble can be dried and optionally coated with one or more topical coatings comprising, e.g., flavors, fats, oils, powdered ingredients, and the like. Kibbles may also be prepared from dough by baking, rather than extrusion, in which the dough is placed into a mold before dry-heat processing.

In preparing a composition, any ingredient generally may be incorporated into the composition during the processing of the formulation, e.g., during and/or after mixing of the other components of the composition. Distribution of these components into the composition can be accomplished by conventional means. In certain embodiments, ground animal and/or poultry proteinaceous tissues are mixed with other ingredients, including nutritional balancing agents, inorganic salts, and may further include cellulose, beet pulp, bulking agents and the like, along with sufficient water for processing.

In accordance with another aspect, provided is a method for alleviating, mitigating, or reducing symptoms of a pet suffering from a renal disease. The method may comprise increasing the ratio of circulating albumin to globulin in the pet by administering a pet food composition disclosed herein to the pet. In some instances, the method may include providing and/or feeding the canine the pet food compositions for 1 or more days, preferably 5 or more days, preferably 7 or more days, preferably 10 or more days, preferably 14 or more days, preferably 30 or more days, or preferably 42 or more days. The method may include feeding the pet one time a day, two times a day, three times a day, or in some embodiments four or more times a day.

Preferably, the method produces an increase of about 5% or more, about 10% or more, about 15% or more, about 20% or more, or about 25% or more, of at least one long-chain acylcarnitine, osmotic regulator, phospholipid, sphingolipid, endocannabinoid, the incorporation of anti-inflammatory long chain polyunsaturated docosahexaenoate (22:6n3) into phospholipids, and combinations of two or more thereof. The increase in the one or more of long-chain acylcarnitines, osmotic regulators, phospholipids, sphingolipids, endocannabinoids, and/or the anti-inflammatory long chain polyunsaturated docosahexaenoate (22:6n3) into phospholipids may be achieved after about 3 days post-ingestion of the pet consuming such pet food composition at least once a day (e.g., twice or three times a day) based on the desired and/or recommended daily nutritional intake for the pet for 14 days.

The methods may also increase the ratio of circulating albumin to globulin in a pet consuming the pet food composition. In some embodiments, the method may increase the ratio of circulating albumin to globulin by about 3% or more, about 6% or more, about 9% or more, about 14% or more, about 17% or more, about 22% or more, about 27% or more. The increase in the ratio of circulating albumin to globulin may be achieved after about 3 days post-ingestion by a pet consuming such pet food composition at least once a day (e.g., twice or three times a day) based on the desired and/or recommended daily nutritional intake for the pet for 14 days.

Preferably, the method maintains both a blood urea concentration and a creatinine concentration within a 10±% range (e.g., within a 7±% range or 5±% range) after about 3 days post-ingestion by a pet, such as a being fed the pet food composition at least once a day (e.g., twice or three times a day) based on the desired and/or recommended daily nutritional intake for the pet for 14 days. As noted above, the recommended daily nutritional intake may be based on the Association of American Feed Control Officials (AAFCO) recommendations for the pet.

According to another aspect, provided is a method for alleviating, mitigating, or reducing symptoms of a pet suffering from a renal disease comprising: diagnosing a pet with renal disease by measuring serum albumin in the pet; and increasing the ratio of circulating albumin to globulin in the pet by administering an amount of a pet food composition disclosed herein to the pet. In some embodiments, the method further comprises measuring serum albumin in the pet after one or more weeks of the pet being administered the amount of pet food composition, and instructing an increase or decrease in the amount of pet food composition administered to the pet.

### EXAMPLES

### Example 1

A non-limiting, exemplary pet food composition (Example Composition A) and a comparative pet food composition (Comparative Composition 1) were prepared in accordance with aspects of the invention. The formula of Example Composition A and Comparative Composition 1 are shown in Table 1, provided below. Table 2 provides a summary of the nutritional values of Example Composition A and Comparative Composition 1. Table 2 also provides the percentage difference between the nutritional value of Example Composition A and Comparative Composition 1.

TABLE 1

| | Comp. 1 | Ex. A |
|---|---|---|
| Ingredient | (wt. %) | (wt. %) |
| Brown Rice | 17-20 | 17-20 |
| Corn gluten meal and Wheat | 21.5 | 21.5 |
| gluten | | |
| Ground Fresh Chicken | 11.5-13 | 11.5-13 |
| Whole red wheat | 9.5-11 | 9.5-11 |
| C8:0 Medium Chain | | 6 - 10 |
| Triglyceride | | |
| Pork Fat | 12.3 | 2.3 - 6.3 |
| Pearl barley | 7-9.5 | 7-9.5 |
| Dried egg | 2.5-4 | 2.5-4 |
| Pea protein | 2.5-4 | 2.5-4 |
| Palatant | 2.5-3.5 | 2.5-3.5 |
| Fish oil | 1.2-2 | 1.2-2 |
| Lactic acid (84%) | 1-1.5 | 1-1.5 |
| Potassium citrate and | 0.8-1.4 | 0.8-1.4 |
| Potassium chloride | | |
| Calcium sulfate and Calcium | 1-1.5 | 1-1.5 |
| carbonate | | |
| L-Lysine, L-Arginine, L- | 1.74 | 1.74 |
| Threonine, Taurine, L- | | |
| Tryptophan, and DL- | | |
| Methionine | | |
| Choline chloride and | 0.2-0.4 | 0.2-0.4 |
| Carnitine | | |
| Vitamin Mix, Mineral Mix, | 0.6 | 0.6 |
| and Vitamin E (50%) | | |
| Sodium chloride (iodized) | 0.1-0.3 | 0.1-0.3 |

TABLE 2

| | | | Percent Difference |
|---|---|---|---|
| | Comp. 1 | Ex. A | between Ex. A and |
| Nutritional Value | (wt. %) | (wt. %) | Comp. 1 |
| Protein Crude | 29.9 | 29.3 | −2% |
| NFE | 37.8 | 37.5 | −1% |
| Fiber Crude | 0.9 | 1 | 11% |
| Ash | 4.4 | 4.2 | −4% |
| Moisture | 6.1 | 6.3 | 2% |
| Fat Crude | 20.8 | 21.8 | 5% |
| Monounsaturated Fatty | 7.7 | 5.2 | −32% |
| Acids | | | |
| Polyunsaturated Fatty | 4.4 | 3.5 | −20% |
| Acids | | | |
| Saturated Fatty Acids | 6.5 | 11 | 69% |
| C08:0 Octanoic (Caprylic) | 0.1 | 6.8 | 5123% |
| C10:0 Decanoic (Capric) | 0.02 | 0.02 | 0% |
| C12:0 Dodecanoic (Lauric) | 0.02 | 0.02 | 0% |

### Example 2

Example Composition A was evaluated to assess the health effects on felines suffering from renal disease in comparison to Comparative Composition 1. Sixteen felines that were diagnosed with renal disease by the colony veterinarian based on circulating levels of creatinine and symmetric dimethylarginine were evaluated in this Example. Each feline consumed Comparative Composition 1 for a period of one week as a pre-feed regimen. The felines were then randomly separated into two groups, with the first group consuming Example Composition A for a period of eight weeks and the second group consuming Comparative Composition 1 for a period of eight weeks as a first feed regimen. After the eight weeks, the first group of felines was fed Comparative Composition 1 for a period of eight weeks and the second group of felines was fed Example Composition A for a period of eight weeks as a second feed regimen. After each of the pre-feed regimen, the first feed regimen, and the second feed regimen, blood samples were obtained from each of the felines. The blood samples were analyzed by dependent samples t-test, with the statistical criteria for significance having endpoints set as a=0.05.

During this experiment, all felines were in spacious indoor rooms with natural light. The felines received behavioral enrichment by interacting with each other, as well as through playtime with caretakers and access to toys. The felines were provided access to food for approximately 21 hours per day and water ad libitum. All felines were immunized against disease, were monitored for parasites, and received routine heartworm preventative. Additionally, all felines were returned healthy to the colony after the test with no adverse reactions.

Example Composition A improved lipid markers by decreasing compound classes that are positively associated with renal disease including long-chain acylcarnitines, osmotic regulators, phospholipids, sphingolipids and endocannabinoids. Increased long chain acylcarnitines are typically observed in renal disease and are indicative of a failure to adequately catabolize fatty acids in the mitochondria. Example Composition A decreased a broad swath of circulating long chain acycarnitines (see Table 3, below), indicating that Example Composition A improved the lipid metabolism at the level of the mitochondria to reduce an important lipid marker of disease risk. The values for the analytes presented in Table 3 are scaled such that the median value measured across all samples was set to 1.0.

TABLE 3

| Analyte | Comp. 1 | Ex. A | P DIFF |
|---|---|---|---|
| cerotoylcarnitine (C26)* | 1.13 ± 0.05 | 0.56 ± 0.04 | 3.20E−08 |
| lignoceroylcarnitine (C24)* | 1.16 ± 0.07 | 0.67 ± 0.04 | 8.26E−07 |
| ximenoylcarnitine (C26:1)* | 1.11 ± 0.06 | 0.75 ± 0.05 | 7.33E−07 |
| behenoylcarnitine (C22)* | 1.13 ± 0.07 | 0.78 ± 0.07 | 5.31E−05 |
| arachidoylcarnitine (C20)* | 1.04 ± 0.06 | 0.75 ± 0.05 | 2.75E−04 |
| nervonoylcarnitine (C24:1)* | 1.04 ± 0.05 | 0.8 ± 0.06 | 3.92E−04 |
| erucoylcarnitine (C22:1)* | 1.03 ± 0.06 | 0.84 ± 0.07 | 0.00184 |
| dihomo-linoleoylcarnitine | 1.08 ± 0.06 | 0.88 ± 0.06 | 0.00904 |
| (C20:2)* | | | |
| eicosenoylcarnitine (C20:1)* | 1.01 ± 0.06 | 0.88 ± 0.07 | 0.0477 |
| adrenoylcarnitine (C22:4)* | 1.07 ± 0.07 | 0.92 ± 0.08 | 0.0282 |
| docosahexaenoylcarnitine | 0.96 ± 0.11 | 0.87 ± 0.1 | 0.32 |
| (C22:6)* | | | |
| arachidonoylcarnitine (C20:4) | 1.12 ± 0.12 | 1.03 ± 0.09 | 0.377 |
| docosapentaenoylcarnitine | 1.05 ± 0.08 | 1.01 ± 0.08 | 0.431 |
| (C22:5n3)* | | | |
| dihomo-linolenoylcarnitine | 1.04 ± 0.09 | 1.07 ± 0.1 | 0.71 |
| (C20:3n3 or 6)* | | | |

Cholesterol and lipoprotein homeostasis is perturbed in renal disease with increased levels of cholesterol sterol, LDL and HDL. Example Composition A also decreased the levels of circulating total cholesterol (see Table 4, below), providing evidence that Example Composition A promoted normalization of dyslipidosis in renal disease.

TABLE 4

| | Serum | Comp. 1 | Ex. A | P |
|---|---|---|---|---|
| Data Type | Analyte | (mg / dL) | (mg / dL) | difference |
| Clinical Data | Cholesterol | 205.4 ± 12.8 | 155.4 ± 10.2 | <0.0001 |
| Metabolomic | Cholesterol | 1.16 ± 0.07 | 0.87 ± 0.06 | 6.89E−05 |
| Data | Cholesterol | 1.14 ± 0.10 | 0.77 ± 0.06 | 2.18E−08 |
| | Sulfate | | | |

Phospholipids and sphingolipids generally increase in pets suffering from renal disease. Phospholipids and sphingolipids can be markers of cell membrane damage/turnover as well as inflammation, respectively. Example Composition A decreased three separate classes of phospholipids—namely, the glycerophosphocholine, glycerophosphoethanolamine, glycerophosphoinositol classes of phospholipids. Notably, these classes of phospholipids were uniformly decreased, such that the vast majority of these phospholipids significantly decreased with only a few phospholipids in these classes increasing (see Tables 5-7, below). Additionally, there was a decrease in every of the osmolyte congeners for these phospholipid classes. Because these osmolyte congeners are increased by hyperosmotic kidney failure and kidney damage, it is generally believed that the decrease in these osmolyte congeners corresponds to a health benefit provided by Example Composition A, which complements the decrease in phospholipids. The values for the analytes presented in Tables 5-7 are scaled such that the median value measured across all samples was set to 1.0.

TABLE 5

| Analyte | Comp. 1 | Ex. A | P DIFF |
|---|---|---|---|
| 1-linoleoyl-2-linolenoyl-GPC (18:2/18:3)* | 1.19 ± 0.1 | 0.79 ± 0.08 | 0.000003 |
| 1,2-dilinoleoyl-GPC (18:2/18:2) | 1.17 ± 0.06 | 0.79 ± 0.04 | 1.86E−08 |
| 1-palmitoleoyl-2-linolenoyl-GPC | 1.2 ± 0.12 | 0.85 ± 0.11 | 0.000056 |
| (16:1/18:3)* | | | |
| 1,2-dilinolenoyl-GPC (18:3/18:3)* | 1.19 ± 0.1 | 0.89 ± 0.12 | 0.01 |
| 1-palmitoyl-2-stearoyl-GPC (16:0/18:0) | 1.23 ± 0.11 | 0.88 ± 0.08 | 0.000046 |
| 1-(1-enyl-palmitoyl)-2-linoleoyl-GPC | 1.08 ± 0.04 | 0.83 ± 0.06 | 0.00026 |
| (P-16:0/18:2)* | | | |
| 1-lignoceroyl-GPC (24:0) | 1.12 ± 0.09 | 0.88 ± 0.05 | 0.00045 |
| 1-palmitoyl-2-arachidonoyl-GPC | 1.06 ± 0.03 | 0.86 ± 0.03 | 0.000032 |
| (16:0/20:4n6) | | | |
| 1-arachidonoyl-GPC (20:4n6)* | 1.12 ± 0.05 | 0.91 ± 0.05 | 0.0006 |
| 1-linoleoyl-GPC (18:2) | 1.09 ± 0.04 | 0.9 ± 0.03 | 0.0000054 |
| 1-(1-enyl-palmitoyl)-2-arachidonoyl- | 1.07 ± 0.05 | 0.9 ± 0.07 | 0.004 |
| GPC (P-16:0/20:4)* | | | |
| 1,2-dipalmitoyl-GPC (16:0/16:0) | 1.09 ± 0.06 | 0.91 ± 0.04 | 0.00092 |
| glycerophosphorylcholine (GPC) | 1.03 ± 0.06 | 0.86 ± 0.04 | 0.01 |
| 1-palmitoyl-2-oleoyl-GPC (16:0/18:1) | 1.04 ± 0.03 | 0.89 ± 0.03 | 0.00038 |
| 1-palmitoyl-2-linoleoyl-GPC (16:0/18:2) | 1.02 ± 0.03 | 0.88 ± 0.03 | 0.00035 |
| 1-(1-enyl-palmitoyl)-GPC (P-16:0)* | 1.08 ± 0.05 | 0.94 ± 0.05 | 0.0032 |
| 1-oleoyl-GPC (18:1) | 1.07 ± 0.04 | 0.92 ± 0.03 | 0.000056 |
| 1-(1-enyl-palmitoyl)-2-palmitoyl-GPC | 1.01 ± 0.02 | 0.88 ± 0.04 | 0.01 |
| (P-16:0/16:0)* | | | |
| 1-oleoyl-2-linoleoyl-GPC (18:1/18:2)* | 1.09 ± 0.04 | 0.96 ± 0.04 | 0.000042 |
| 1-palmitoleoyl-2-linoleoyl-GPC | 1.08 ± 0.04 | 0.96 ± 0.04 | 0.0046 |
| (16:1/18:2)* | | | |
| 1-palmitoyl-GPC (16:0) | 1.01 ± 0.03 | 0.9 ± 0.03 | 0.00017 |
| 2-palmitoyl-GPC (16:0)* | 1.08 ± 0.09 | 0.95 ± 0.06 | 0.2 |
| 1-linolenoyl-GPC (18:3)* | 1.05 ± 0.08 | 0.95 ± 0.08 | 0.05 |
| 2-palmitoleoyl-GPC (16:1)* | 1.08 ± 0.14 | 1.02 ± 0.15 | 0.57 |
| 1-stearoyl-GPC (18:0) | 1.03 ± 0.03 | 0.95 ± 0.03 | 0.02 |
| 1-palmitoleoyl-GPC (16:1)* | 1.12 ± 0.05 | 1.05 ± 0.07 | 0.11 |
| 1-palmitoyl-2-palmitoleoyl-GPC (16:0/16:1)* | 1.06 ± 0.04 | 0.98 ± 0.04 | 0.05 |
| 1-stearoyl-2-linoleoyl-GPC (18:0/18:2)* | 1.04 ± 0.02 | 0.97 ± 0.03 | 0.01 |
| 1-stearoyl-2-arachidonoyl-GPC (18:0/20:4) | 0.98 ± 0.03 | 0.92 ± 0.03 | 0.07 |
| 1-(1-enyl-palmitoyl)-2-oleoyl-GPC | 1.03 ± 0.04 | 1.03 ± 0.08 | 0.55 |
| (P-16:0/18:1)* | | | |
| 1-stearoyl-2-oleoyl-GPC (18:0/18:1) | 1.01 ± 0.03 | 1 ± 0.03 | 0.7 |
| 1-myristoyl-2-arachidonoyl-GPC | 1.09 ± 0.07 | 1.13 ± 0.1 | 0.87 |
| (14:0/20:4)* | | | |
| 1-myristoyl-2-palmitoyl-GPC | 1.01 ± 0.05 | 1.03 ± 0.05 | 0.64 |
| (14:0/16:0) | | | |
| 1-palmitoyl-2-docosahexaenoyl-GPC | 0.96 ± 0.03 | 1.01 ± 0.05 | 0.38 |
| (16:0/22:6) | | | |
| 1-oleoyl-2-docosahexaenoyl-GPC | 1.02 ± 0.05 | 1.1 ±0.05 | 0.14 |
| (18:1/22:6)* | | | |
| 1-stearoyl-2-docosahexaenoyl-GPC | 0.99 ± 0.04 | 1.1 ± 0.04 | 0.01 |
| (18:0/22:6) | | | |
| 1,2-distearoyl-GPC (18:0/18:0) | 0.63 ± 0.1 | 2.4 ± 0.33 | 0.0000052 |

TABLE 6

| Analyte | Comp. 1 | Ex. A | P DIFF |
|---|---|---|---|
| 1-oleoyl-GPE (18:1) | 1.32 ± 0.11 | 0.79 ± 0.07 | 0.00000051 |
| 1-palmitoyl-2-oleoyl-GPE (16:0/18:1) | 1.05 ± 0.18 | 0.63 ± 0.07 | 0.013 |
| 1-(1-enyl-palmitoyl)-2-arachidonoyl-GPE | 1.06 ± 0.05 | 0.78 ± 0.06 | 0.00004 |
| (P-16:0/20:4)* | | | |
| 1-(1-enyl-stearoyl)-2-linoleoyl-GPE | 1.15 ± 0.1 | 0.85 ± 0.06 | 0.0000064 |
| (P-18:0/18:2)* | | | |
| 1-(1-enyl-palmitoyl)-GPE (P-16:0)* | 1.06 ± 0.03 | 0.84 ± 0.03 | 0.00001 |
| 1-(1-enyl-stearoyl)-2-oleoyl-GPE | 1.13 ± 0.12 | 0.88 ± 0.08 | 0.00033 |
| (P-18:0/18:1) | | | |
| 1-(1-enyl-stearoyl)-GPE (P-18:0)* | 1.09 ± 0.05 | 0.87 ± 0.05 | 0.000032 |
| 1-(1-enyl-stearoyl)-2-arachidonoyl-GPE | 1.06 ± 0.05 | 0.86 ± 0.06 | 0.002 |
| (P-18:0/20:4)* | | | |
| 1-(1-enyl-palmitoyl)-2-oleoyl-GPE | 1.1 ± 0.06 | 0.87 ± 0.04 | 0.00084 |
| (P-16:0/18:1)* | | | |
| 1-linoleoyl-GPE (18:2)* | 1.16 ± 0.06 | 0.95 ± 0.05 | 0.0003 |
| 1-(1-enyl-oleoyl)-GPE (P-18:1)* | 1.06 ± 0.05 | 0.9 ± 0.06 | 0.0089 |
| 1-arachidonoyl-GPE (20:4n6)* | 1.13 ± 0.05 | 0.99 ± 0.05 | 0.0029 |
| 1-palmitoyl-2-arachidonoyl-GPE | 1.12 ± 0.1 | 1 ± 0.09 | 0.37 |
| (16:0/20:4)* | | | |
| 1-palmitoyl-GPE (16:0) | 1.05 ± 0.06 | 0.96 ± 0.06 | 0.054 |
| 1-stearoyl-GPE (18:0) | 1.01 ± 0.04 | 1.02 ± 0.05 | 0.98 |
| 1-palmitoyl-2-linoleoyl-GPE (16:0/18:2) | 0.9 ± 0.11 | 0.91 ± 0.13 | 0.94 |
| 1-stearoyl-2-oleoyl-GPE (18:0/18:1) | 1.38 ± 0.21 | 1.45 ± 0.23 | 0.83 |
| 2-stearoyl-GPE (18:0)* | 1.05 ± 0.09 | 1.13 ± 0.08 | 0.29 |
| 1-stearoyl-2-linoleoyl-GPE (18:0/18:2)* | 1.04 ± 0.09 | 1.25 ± 0.1 | 0.02 |
| 1-stearoyl-2-arachidonoyl-GPE (18:0/20:4) | 1.03 ± 0.09 | 1.35 ± 0.11 | 0.00044 |
| 1-palmitoyl-2-docosahexaenoyl-GPE | 0.84 ± 0.07 | 1.21 ± 0.1 | 0.00055 |
| (16:0/22:6)* | | | |
| 1-stearoyl-2-docosahexaenoyl-GPE | 0.75 ± 0.05 | 1.4 ± 0.12 | 0.000062 |
| (18:0/22:6)* | | | |
| glycerophosphoethanolamine | 1.01 ± 0.04 | 0.92 ± 0.04 | 0.018 |

TABLE 7

| Analyte | Comp. 1 | Ex. A | P DIFF |
|---|---|---|---|
| 1-palmitoyl-2-arachidonoyl-GPI | 1.18 ± 0.12 | 0.74 ± 0.07 | 0.00001 |
| (16:0/20:4)* | | | |
| 1-palmitoyl-2-oleoyl-GPI (16:0/18:1)* | 1.22 ± 0.13 | 0.87 ± 0.07 | 0.0002 |
| 1-palmitoyl-2-linoleoyl-GPI (16:0/18:2) | 1.01 ± 0.05 | 0.76 ± 0.07 | 0.00033 |
| 1-stearoyl-2-oleoyl-GPI (18:0/18:1)* | 1.23 ± 0.12 | 0.83 ± 0.07 | 0.00059 |
| 1-arachidonoyl-GPI (20:4)* | 1.16 ± 0.07 | 0.87 ± 0.04 | 0.0012 |
| 1-oleoyl-GPI (18:1) | 1.51 ± 0.24 | 0.76 ± 0.11 | 0.0022 |
| 1-stearoyl-2-arachidonoyl-GPI (18:0/20:4) | 0.98 ± 0.03 | 0.92 ± 0.03 | 0.02 |
| 1-palmitoyl-GPI (16:0) | 1.27 ± 0.18 | 0.88 ± 0.08 | 0.028 |
| 1-stearoyl-2-linoleoyl-GPI (18:0/18:2) | 1.08 ± 0.1 | 0.9 ± 0.06 | 0.03 |
| 1-linoleoyl-GPI (18:2)* | 1.26 ± 0.15 | 0.98 ± 0.06 | 0.13 |
| 1-stearoyl-GPI (18:0) | 1.1 ± 0.12 | 0.97 ± 0.06 | 0.46 |
| glycerophosphoinositol* | 1.04 ± 0.05 | 0.87 ± 0.05 | 0.053 |

Example Composition A also decreased sphingolipids related to inflammation; notably nearly all of the changes were significant decreases (see Table 8, below). Intriguingly, among the very few increases in phospholipid compounds was that of phospholipids containing 22:6n3 (see Tables 3 and 4). Phospholipids n3PUFA (such as, 22:6n3) can have an anti-inflammatory effect through the production of series 3 prostanoids. This was unexpected since Comparative Composition 1 as well as Example Composition A contained similar level of 22:6n3 fatty acids by analysis—specifically, 0.18 wt. % for Comparative Composition 1 and 0.19 wt. % for Example Composition A. The values for the analytes presented in Table 8 are scaled such that the median value measured across all samples was set to 1.0.

TABLE 8

| Analyte | Comp. 1 | Ex. A | P DIFF |
|---|---|---|---|
| behenoyl dihydrosphingomyelin (d18:0/22:0)* | 1.32 ± 0.14 | 0.92 ± 0.13 | 0.00027 |
| behenoyl sphingomyelin (d18:1/22:0)* | 1.29 ± 0.13 | 0.96 ± 0.08 | 0.033 |
| glycosyl-N-(2-hydroxynervonoyl)-sphingosine | 1.04 ± 0.09 | 0.93 ± 0.13 | 0.37 |
| (d18:1/24:1(2OH))* | | | |
| glycosyl-N-behenoyl-sphingadienine (d18:2/22:0)* | 1.14 ± 0.12 | 0.82 ± 0.1 | 0.0007 |
| glycosyl-N-nervonoyl-sphingosine (d18:1/24:1)* | 1.04 ± 0.1 | 0.8 ± 0.09 | 0.036 |
| glycosyl-N-palmitoyl-sphingosine (d18:1/16:0) | 1.06 ± 0.05 | 0.81 ± 0.05 | 0.000016 |
| glycosyl-N-stearoyl-sphingosine (d18:1/18:0) | 1.05 ± 0.07 | 0.88 ± 0.07 | 0.0016 |
| glycosyl-N-tricosanoyl-sphingadienine | 1.2 ± 0.15 | 0.89 ± 0.13 | 0.067 |
| (d18:2/23:0)* | | | |
| heptadecasphingosine (d17:1) | 1.14 ± 0.07 | 1 ± 0.07 | 0.054 |
| hexadecasphingosine (d16:1)* | 1.16 ± 0.11 | 1.05 ± 0.1 | 0.25 |
| hydroxypalmitoyl sphingomyelin | 1.07 ± 0.06 | 0.86 ± 0.06 | 0.0004 |
| (d18:1/16:0(OH)) ** | | | |
| lactosyl-N-stearoyl-sphingosine (d18:1/18:0)* | 1.19 ± 0.1 | 0.97 ± 0.1 | 0.06 |
| lignoceroyl sphingomyelin (d18:1/24:0) | 1.15 ± 0.09 | 0.87 ± 0.06 | 0.0004 |
| myristoyl dihydrosphingomyelin (d18:0/14:0)* | 1 ± 0.06 | 1.18 ± 0.1 | 0.0084 |
| N-behenoyl-sphingadienine (d18:2/22:0)* | 1.12 ± 0.12 | 1.04 ± 0.09 | 0.61 |
| N-palmitoyl-heptadecasphingosine (d17:1/16:0)* | 1.04 ± 0.09 | 0.88 ± 0.06 | 0.03 |
| N-palmitoyl-sphinganine (d18:0/16:0) | 1.17 ± 0.09 | 0.95 ± 0.07 | 0.0045 |
| N-palmitoyl-sphingosine (d18:1/16:0) | 1.12 ± 0.07 | 0.88 ± 0.05 | 0.0008 |
| N-stearoyl-sphinganine (d18:0/18:0)* | 1.12 ± 0.08 | 1.06 ± 0.11 | 0.23 |
| N-stearoyl-sphingosine (d18:1/18:0)* | 1.03 ± 0.06 | 0.92 ± 0.05 | 0.048 |
| palmitoyl dihydrosphingomyelin (d18:0/16:0)* | 1.12 ± 0.07 | 0.8 ± 0.05 | 0.00000015 |
| palmitoyl sphingomyelin (d18:1/16:0) | 1.06 ± 0.03 | 0.85 ± 0.03 | 0.0000056 |
| palmitoyl-sphingosine-phosphoethanolamine | 1.05 ± 0.04 | 0.72 ± 0.03 | 0.000012 |
| (d18:1/16:0) | | | |
| sphingadienine | 0.91 ± 0.09 | 0.93 ± 0.07 | 0.65 |
| sphinganine | 1.14 ± 0.08 | 0.93 ± 0.06 | 0.068 |
| sphinganine-1-phosphate | 1.36 ± 0.17 | 1.22 ± 0.11 | 0.49 |
| sphingomyelin (d17:1/14:0, d16:1/15:0)* | 1.19 ± 0.11 | 1.05 ± 0.08 | 0.016 |
| sphingomyelin (d17:1/16:0, d18:1/15:0, | 1.06 ± 0.05 | 0.88 ± 0.05 | 0.0017 |
| d16:1/17:0)* | | | |
| sphingomyelin (d17:2/16:0, d18:2/15:0)* | 1.1 ± 0.1 | 0.89 ± 0.06 | 0.002 |
| sphingomyelin (d18:0/18:0, d19:0/17:0)* | 1.16 ± 0.08 | 0.81 ± 0.09 | 0.00043 |
| sphingomyelin (d18:0/20:0, d16:0/22:0)* | 1.3 ± 0.14 | 0.96 ± 0.11 | 0.00069 |
| sphingomyelin (d18:1/14:0, d16:1/16:0)* | 1.06 ± 0.05 | 1 ± 0.04 | 0.075 |
| sphingomyelin (d18:1/17:0, d17:1/18:0, d19:1/16:0) | 1.06 ± 0.05 | 0.94 ± 0.05 | 0.041 |
| sphingomyelin (d18:1/18:1, d18:2/18:0) | 1.1 ± 0.05 | 0.94 ± 0.05 | 0.0019 |
| sphingomyelin (d18:1/19:0, d19: 1/18:0)* | 1.03 ± 0.06 | 0.92 ± 0.06 | 0.031 |
| sphingomyelin (d18:1/20:0, d16:1/22:0)* | 1.04 ± 0.05 | 0.91 ± 0.05 | 0.0087 |
| sphingomyelin (d18:1/20:1, d18:2/20:0)* | 1.05 ± 0.06 | 0.96 ± 0.05 | 0.038 |
| sphingomyelin (d18:1/20:2, d18:2/20:1, | 1.01 ± 0.08 | 0.94 ± 0.09 | 0.093 |
| d16:1/22:2)* | | | |
| sphingomyelin (d18:1/21:0, d17:1/22:0, | 1.22 ± 0.11 | 1.03 ± 0.08 | 0.0055 |
| d16:1/23:0)* | | | |
| sphingomyelin (d18:1/22:1, d18:2/22:0, | 1.01 ± 0.05 | 0.97 ± 0.04 | 0.36 |
| d16:1/24:1)* | | | |
| sphingomyelin (d18:1/22:2, d18:2/22:1, | 0.99 ± 0.04 | 1.06 ± 0.07 | 0.172 |
| d16:1/24:2)* | | | |
| sphingomyelin (d18:1/24:1, d18:2/24:0)* | 1.16 ± 0.07 | 0.9 ± 0.06 | 0.0029 |
| sphingomyelin (d18:1/25:0, d19:0/24:1, d20:1/23:0, | 1.12 ± 0.11 | 0.92 ± 0.09 | 0.029 |
| d19:1/24:0)* | | | |
| sphingomyelin (d18:2/14:0, d18:1/14:1)* | 1.04 ± 0.09 | 0.92 ± 0.08 | 0.0043 |
| sphingomyelin (d18:2/16:0, d18:1/16:1)* | 1.07 ± 0.04 | 0.87 ± 0.04 | 0.00015 |
| sphingomyelin (d18:2/18:1)* | 1.12 ± 0.05 | 0.89 ± 0.06 | 0.0013 |
| sphingomyelin (d18:2/21:0, d16:2/23:0)* | 1.03 ± 0.07 | 0.96 ± 0.07 | 0.15 |
| sphingomyelin (d18:2/23:0, d18:1/23:1, | 1.15 ± 0.08 | 0.93 ± 0.06 | 0.002 |
| d17:1/24:1)* | | | |
| sphingomyelin (d18:2/23:1)* | 0.99 ± 0.05 | 0.97 ± 0.05 | 0.7 |
| sphingomyelin (d18:2/24:1, d18: 1/24:2)* | 1.04 ± 0.05 | 0.93 ± 0.05 | 0.025 |
| sphingomyelin (d18:2/24:2)* | 0.96 ± 0.03 | 0.97 ± 0.06 | 0.9 |
| sphingosine 1-phosphate | 1.01 ± 0.05 | 1.19 ± 0.09 | 0.019 |
| stearoyl sphingomyelin (d18:1/18:0) | 1.06 ± 0.05 | 0.95 ± 0.05 | 0.021 |
| tricosanoyl sphingomyelin (d18:1/23:0)* | 1.09 ± 0.09 | 0.83 ± 0.08 | 0.0032 |

Additionally, Example Composition A decreased levels of acylethanolamide endocannabinoids. Without being limited to any particular theory, it is believed that endocannabinoids may be pro-fibrotic and worsen kidney fibrosis. Example Composition A decreased endocannabinoids having CB 1 activity (see Table 9, below), which is believed to provide a benefit to slowing renal failure through maintenance of kidney ultrastructure. The values for the analytes presented in Table 9 are scaled such that the median value measured across all samples was set to 1.0.

TABLE 9

| Type of | | | | P |
|---|---|---|---|---|
| NAAN | Analyte | Comp. 1 | Ex. A | DIFF |
| Ethanolamide | linoleoyl ethanolamide | 1.05 ± 0.05 | 1.04 ± 0.05 | 0.8 |
| | palmitoyl ethanolamide | 1.03 ± 0.03 | 0.94 ± 0.02 | 0.0056 |
| | oleoyl ethanolamide | 1.1 ± 0.06 | 0.98 ± 0.07 | 0.0021 |
| | stearoyl ethanolamide | 1.05 ± 0.04 | 0.92 ± 0.03 | 0.0006 |
| 2-acylglycerol | 2-linoleoylglycerol (18:2) | 1.02 ± 0.13 | 1.01 ± 0.06 | 0.64 |
| choline | docosahexaenoylcholine | 1.2 ± 0.19 | 1.37 ± 0.23 | 0.3 |
| | eicosapentaenoylcholine | 1.66 ± 0.33 | 1.53 ± 0.27 | 0.99 |
| | stearoylcholine* | 1.44 ± 0.26 | 1.3 ± 0.24 | 0.7 |
| | arachidonoylcholine | 1.45 ± 0.25 | 1.23 ± 0.2 | 0.5 |
| | oleoylcholine | 1.58 ± 0.3 | 1.3 ± 0.23 | 0.46 |
| | palmitoylcholine | 1.38 ± 0.23 | 1.14 ± 0.17 | 0.34 |
| | linoleoylcholine* | 1.41 ± 0.23 | 1.1 ± 0.18 | 0.2 |
| taurine | N-linoleoyltaurine* | 0.91 ± 0.11 | 1.04 ± 0.08 | 0.059 |
| | hexanoyltaurine | 0.94 ± 0.06 | 1.11 ± 0.09 | 0.037 |
| | N-linolenoyltaurine* | 0.84 ± 0.1 | 0.89 ± 0.1 | 0.39 |
| | N-stearoyltaurine | 1.1 ± 0.15 | 1.11 ± 0.07 | 0.43 |
| | N-palmitoyltaurine | 1.05 ± 0.11 | 1.02 ± 0.07 | 0.93 |
| | N-arachidonoyltaurine | 0.97 ± 0.17 | 0.87 ± 0.11 | 0.7 |
| | N-myristoyltaurine* | 1.04 ± 0.09 | 0.97 ± 0.09 | 0.37 |
| serine | N-stearoylserine* | 1.11 ± 0.12 | 1.18 ± 0.1 | 0.51 |
| | N-oleoylserine | 1.18 ± 0.13 | 1.12 ± 0.09 | 0.91 |
| | N-palmitoylserine | 1.08 ± 0.1 | 0.98 ± 0.08 | 0.35 |
| | N-linoleoylserine* | 1.14 ± 0.17 | 1.03 ± 0.14 | 0.4 |
| glycine | N-palmitoylglycine | 1.05 ± 0.08 | 1.01 ± 0.06 | 0.8 |
| | N-octanoylglycine | 0.8 ± 0.12 | 0.78 ± 0.16 | 0.51 |
| | hexanoylglycine | 1.1 ± 0.11 | 0.96 ± 0.12 | 0.17 |
| | N-linoleoylglycine | 1.21 ± 0.15 | 0.96 ± 0.08 | 0.17 |

Example Composition A had a beneficial effect to alter the disposition of lipid catabolism toward the circulating ketone energy substrate, β-hydroxybutyrate. Table 10 shows that the levels of this lipid catabolite were increased by Example Composition A. β-hydroxybutyrate has pronounced anti-inflammatory and renal protective benefits, thus further strengthening the potency of Example Composition A for subjects with renal disease.

TABLE 10

| | Comp. 1 | Ex. A | |
|---|---|---|---|
| Analyte | (umol/L) | (umol/L) | P DIFF |
| b-hydroxybutyrate | 121.6 ± 7.8 | 148.8± 12.1 | 0.007 |

In addition to the improved amounts of several classes of lipid markers for renal disease, Example Composition A improved an important clinical marker of renal disease outcome. Specifically, the ratio of albumin to globulin (“A:G Ratio”) was increased by Example Composition A, with the increase being the result of an increase in albumin and a decrease in globulin (see Table 11). This indicates that Example Composition A works by multiple mechanisms including improved renal integrity (decreased loss of albumin through proteinuria) and decreased inflammatory status (reduced production of immunoglobulins). Surprisingly, even though Example Composition A increased albumin, a marker of protein replete status, it did not increase blood urea or blood creatinine—which indicates a selective property of Example Composition A to support protein anabolism without over-provisioning of protein allocation toward urea or creatinine (see Table 12). Additionally, as seen in Table 11, Example Composition A increased the ketone levels (e.g., B-HBA) relative to Comparative Composition 1, which indicates an improvement in the pets' metabolism.

TABLE 11

| Analyte | Comp. 1 | Ex. A | P DIFF |
|---|---|---|---|
| Alb/Glob Ratio | 0.88 ± 0.05 | 0.94 ± 0.05 | 0.045 |
| Albumin (g / dL) | 3.13 ± 0.06 | 3.25 ± 0.06 | 0.082 |
| Globulin (g / dL) | 3.69 ± 0.17 | 3.56 ± 0.17 | 0.029 |
| B-HBA (mg / dL) | 1.27 ± 0.08 | 1.55 ± 0.13 | 0.008 |

TABLE 12

| | Comp. 1 | Ex. A | |
|---|---|---|---|
| Analyte | (mg / dL) | (mg / dL) | P DIFF |
| BUN | 25 ± 1.5 | 25.4 ± 1.2 | 0.927 |
| Creatinine | 1.56 ± 0.07 | 1.59 ± 0.08 | 0.530 |

### Example 3

A non-limiting, exemplary pet food composition (Example Composition B) and a comparative pet food composition (Comparative Composition 2) were prepared in accordance with aspects of the invention. The nutritional values of Example Composition B and Comparative Composition 2 are shown in Table 13, provided below. Table 13 also provides the percentage difference between the nutritional value of Example Composition B and Comparative Composition 2.

TABLE 13

| | | | Percent |
|---|---|---|---|
| | | | Difference |
| | Comp. 2 | Ex. B | between Ex. B |
| Nutritional Value | (wt. %) | (wt. %) | and Comp. 2 |
| Protein Crude | 49.5 | 35.9 | −27% |
| NFE | 15.6 | 7.6 | −51% |
| Fiber Crude | 4.1 | 5.6 | 37% |
| Ash | 5.9 | 6.98 | 18% |
| Moisture | 3.9 | 7.19 | 87% |
| Fat Crude | 21.1 | 36.7 | 74% |
| Monounsaturated Fatty | 8.23 | 9.83 | 19% |
| Acids | | | |
| Polyunsaturated Fatty Acids | 4.48 | 5.22 | 17% |
| Saturated Fatty Acids | 5.53 | 13.71 | 148% |
| C08:0 Octanoic (Caprylic) | 0.02 | 6.97 | 34750% |
| C10:0 Decanoic (Capric) | 0.02 | 0.02 | 0% |
| C12:0 Dodecanoic (Lauric) | 0.02 | 0.02 | 0% |

The amount of medium chain triglyceride having an octanoate component in Example Composition B and Comparative Composition 2 was determined as a percent of grams per kilocalorie and as a weight percentage relative to the overall pet food composition, as shown in Table 14.

TABLE 14

| | Units of Measure | Comp. 2 | Ex. B |
|---|---|---|---|
| Total Energy | kcals/kg | 3963 | 4410 |
| Total Fat | Wt. % | 21.1 | 36.7 |
| Total Fat | g/1000 kcal | 53.2 | 83.2 |
| C8 Octanoate | Wt. % | 0.02 | 6.97 |
| C8 Octanoate | g/1000 kcal | 0.1 | 15.8 |
| C8 Octanoate | % of total fat | 0.1 | 19.0 |

### Example 4

Example Composition B was evaluated to assess the health effects on felines suffering from renal disease in comparison to Comparative Composition 2 according to the procedures employed in Example 2. A summary of the results, specifically regarding to B-HBA, albumin, globulin, the ratio of albumin to globulin, and hexanoytaurine are show in Table 15.

TABLE 15

| | | | P difference |
|---|---|---|---|
| | | | (Comp. 2 compared |
| Analysis | | | to Ex. B by Linear |
| Columns | Comp. 2 | Ex. B | Mixed Model) |
| Subject Number | 39 | 37 | |
| B-HBA (mg/dL) | 1.07 ± 0.05 | 2.05 ± 0.18 | 8.2E−07 |
| Albumin/ | 1.05 ± 0.03 | 1.24 ± 0.04 | 3.6E−11 |
| Globulin Ratio | | | |
| Albumin (g/dL) | 3.42 ± 0.05 | 3.56 ± 0.06 | 0.002 |
| Globulin (g/dL) | 3.34 ± 0.08 | 2.96 ± 0.09 | 1.2E−10 |
| Hexanoyltaurine | −0.21 ± 0.07 | 0.19 ± 0.07 | 5.2E−06 |
| (Log2(Median | | | |
| Centered Relative | | | |
| Fold Value)) | | | |

As seen in Table 15, Example Composition B improved an important clinical marker of renal disease outcome. Specifically, the ratio of albumin to globulin (“A:G Ratio”) was increased by Example Composition B, with the increase being the result of an increase in albumin and a decrease in globulin. This indicates that Example Composition B, similar to Example Composition A, works by multiple mechanisms including improved renal integrity (decreased loss of albumin through proteinuria) and decreased inflammatory status (reduced production of immunoglobulins).

1-33. (canceled)

34. A pet food composition comprising:
from about 5 g/1000 kcal to about 80 g/1000 kcal of a fat, the fat comprising one or more medium chain triglyceride, wherein at least one medium chain triglyceride comprises an octanoate component, the medium chain triglyceride comprising the octanoate component comprising about 7% or more of the total weight of fat in the pet food composition;
from about 40 g/1000 kcal to about 150 g/1000 kcal of a protein;
optionally, from about 5 g/1000 kcal to about 55 g/1000 kcal of a fiber; and
optionally, from about 3 g/1000 kcal to about 130 g/1000 kcal of a carbohydrate,
wherein all metabolizable energy amounts are based on the total metabolizable energy of the pet food composition.

35. The pet food composition according to claim 34, wherein the fat comprises at least one medium chain triglyceride comprising an octanoate component in an amount of about 12 to about 60 wt. %, based on the total weight of fat in the pet food composition.

36. The pet food composition according to claim 34, wherein the at least one additional medium chain triglyceride has an aliphatic carbon chain component that is decanoate.

37. The pet food composition according to claim 36, wherein the amount of medium chain triglyceride having a decanoate component is about 1 wt. % or less of the total weight of fat in the pet food composition.

38. The pet food composition according to claim 34, wherein pet food composition has a weight ratio of the medium chain triglyceride comprising the octanoate component to a medium chain triglyceride comprising a decanoate component of from about 250:1 to about 500:1.

39. The pet food composition according to claim 34 further comprising one or more fatty acid selected from omega-3 fatty acids, omega-6 fatty acids, lauric acid, myristic acid, palmitic acid, palmitoleic acid, margaric acid, margaroleic acid, stearic acid, oleic acid, stearidonic acid, gadoleic acid, behenic acid, erucic acid, docosatetra acid, and a combination of two or more thereof.

40. The pet food composition according to claim 36, wherein the fat comprises about 6 to about 65 wt. % of one or more fatty acid, based on the total weight of the fat.

41. The pet food composition according to claim 39, wherein the one or more fatty acid comprises an omega-3 fatty acid, an omega-6 fatty acid, or a combination of two or more thereof.

42. The pet food composition of claim 41, wherein the one or more fatty acid comprises an omega-3 fatty acid in an amount of about 1 to about 20 wt. %, based on the total weight of the fat.

43. The pet food composition of claim 41, wherein the one or more fatty acid comprises an omega-6 fatty acid in an amount of about 5 to about 40 wt. %, based on the total weight of the fat.

44. The pet food composition according to claim 41, comprising an omega-3 fatty acid selected from linolenic acid, stearidonic acid, eicosatetraenoic acid, eicosapentaenoic acid, docosapentaenoic acid, docosahexaenoic acid, and a combination of two or more thereof.

45. The pet food composition according to claim 41, comprising an omega-6 fatty acid selected from linoleic acid, calendic acid, eicosadienoic acid, arachidonic acid, docosadienoic acid, adrenic acid, osbond acid, tetracosatetraenoic acid, tetracosapentaenoic acid, and a combination of two or more thereof.

46. The pet food composition according to any of claim 41, wherein the pet food composition has a weight ratio of the omega-3 fatty acids to the omega-6 fatty acids of from about to about 1:20.

47. The pet food composition according to claim 34, wherein the pet food composition produces, after about 3 days post-ingestion, an increase in the ratio of circulating albumin to circulating globulin by about 3% or more.

48. The pet food composition according to claim 34, wherein the pet food composition produces, after about 3 days post-ingestion, an increase of about 5% or more of one or more of long-chain acylcarnitines, osmotic regulators, phospholipids, sphingolipids, endocannabinoids, and a combination of two or more thereof.

49. The pet food composition according to claim 34, wherein the pet food composition produces, after about 3 days post-ingestion, an increase of about 5% or more in the incorporation of the anti-inflammatory long chain polyunsaturated docosahexaenoate into phospholipids.

50. The pet food composition according to claim 34, wherein the pet food composition maintains both a blood urea concentration and a creatinine concentration within a 10±% range.

51. A feline food composition comprising:
a fat comprising one or more medium chain triglyceride, wherein at least one medium chain triglyceride comprises an octanoate component, wherein the at least one medium chain triglyceride comprising the octanoate component comprises about 7 wt. % or more of the fat, based on the total weight of the fat on a dry matter basis;
from about 40 g/1000 kcal to about 150 g/1000 kcal of a protein;
from about 5 g/1000 kcal to about 55 g/1000 kcal of a fiber; and
from about 3 g/1000 kcal to about 130 g/1000 kcal of a carbohydrate,
wherein the pet food composition produces, after about 3 days post-ingestion, an increase in the ratio of circulating albumin to circulating globulin by about 3% or more, and all metabolizable energy amounts are based on the total metabolizable energy of the pet food composition.

52. A method for alleviating, mitigating, or reducing symptoms of a pet suffering from a renal disease, the method comprising: increasing the ratio of circulating albumin to globulin in the pet by administering a pet food composition according to claim 34 to the pet.

53. The method according to claim 52, wherein the method maintains both a blood urea concentration and a creatinine concentration within a 10±% range.
